SEQUENCE LISTING (22 sequences)

SEQ ID NO: 1 - DNA, 1468 bp, Omphalotus olearius
  ophMA (1)..(1468): chromsomal DNA
atgagaggatcgcatcaccatcaccatcacggatccctgaacgacatcttcgaagctcagaaaatcgaatggaaaaaggtcgacgagaacttgtatttccaggagacttccactcagaccaaagctggctcactcaccatcgtcggtaccggtatcgagagtatcggacaaatgacgcttcaggcgttgtcctacatcgaagccgccgcgaaggttttctactgcgtcatcgaccccgccactgaggcattcatcctcaccaagaacaagaactgcgttgacttgtatcagtattacgacaatggcaagtccagattgaacacttacacccaaatgtcagaggtaagcttcgtacaccctcaacagtcgcccacaccccgacgctgacacacgcatagctcatggtcagggaagtccgcaagggcctcgatgtcgtgggcgtcttctacggccacccaggagtgttcgtgaacccgtctcaccgagctctggctatcgccaagagtgaaggctaccgagcgaggatgcttccaggcgtgtctgcggaagattgtctcttcgcggacttgtgtatcgatccttcgaacccgggttgcctgacctacgaggcatcggatttcctgatcagggatcgcccggtcagcatccacagtcacttggtcctgttccaagtcggatgcgtcggtattgctgactttaacttcactggattcgacgtaagttctttcactccaatagctcaagatcttccgcttacctcctcacgaatggccagaacaacaaattcggcgttctcgtcgaccgtctcgagcaagaatacggcgccgagcaccctgtcgtccattacatcgcagctatgatgccacaccaagacccagtcaccgataagtacaccgtcgcgcagctccgtgagcccgagatcgcgaagcgtgttggcggtgtctcgactttctacattcctcccaaggccaggaaagcatcgaacttggacatcataaggcgcctagagctcttgcctgctgggcaagttcccgacaagaaagcgcgtatttacccggccaaccagtgggagcccgatgttcccgaagtcgaaccctacagaccatctgaccaggctgccatcgctcagttggctgaccacgctcctcctgagcaatatcaacctctcgctacttcgaaagccatgtctgatgttatgacgaagttggctttggatccaaaggcactcgccgactacaaagctgatcaccgcgcctttgctcaatctgtccccgacttgacgcctcaggagcgtgcggctttggagctcggtgattcgtgggctattcgttgcgcgatgaagaatatgccctcgtcgctcttggacgctgctcgtgaatccggcgaagaggcatcccaaaacggtttcccatgggtcatcgtcgttggtgtcatcggtgtcatcggatcagtcatgagcacggaataa

SEQ ID NO: 2 - DNA, 1257 bp, Omphalotus olearius
  ophMA_cDNA (1)..(1257)
atgggcgagacttccactcagaccaaagctggctcactcaccatcgtcggtaccggtatcgagagtatcggacaaatgacgcttcaggcgttgtcctacatcgaagccgccgcgaaggttttctactgcgtcatcgaccccgccactgaggcattcatcctcaccaagaacaagaactgcgttgacttgtatcagtattacgacaatggcaagtccagattgaacacttacacccaaatgtcagagctcatggtcagggaagtccgcaagggcctcgatgtcgtgggcgtcttctacggccacccaggagtgttcgtgaacccgtctcaccgagctctggctatcgccaagagtgaaggctaccgagcgaggatgcttccaggcgtgtctgcggaagattgtctcttcgcggacttgtgtatcgatccttcgaacccgggttgcctgacctacgaggcatcggatttcctgatcagggatcgcccggtcagcatccacagtcacttggtcctgttccaagtcggatgcgtcggtattgctgactttaacttcactggattcgacaacaacaaattcggcgttctcgtcgaccgtctcgagcaagaatacggcgccgagcaccctgtcgtccattacatcgcagctatgatgccacaccaagacccagtcaccgataagtacaccgtcgcgcagctccgtgagcccgagatcgcgaagcgtgttggcggtgtctcgactttctacattcctcccaaggccaggaaagcatcgaacttggacatcataaggcgcctagagctcttgcctgctgggcaagttcccgacaagaaagcgcgtatttacccggccaaccagtgggagcccgatgttcccgaagtcgaaccctacagaccatctgaccaggctgccatcgctcagttggctgaccacgctcctcctgagcaatatcaacctctcgctacttcgaaagccatgtctgatgttatgacgaagttggctttggatccaaaggcactcgccgactacaaagctgatcaccgcgcctttgctcaatctgtccccgacttgacgcctcaggagcgtgcggctttggagctcggtgattcgtgggctattcgttgcgcgatgaagaatatgccctcgtcgctcttggacgctgctcgtgaatccggcgaagaggcatcccaaaacggtttcccatgggtcatcgtcgttggtgtcatcggtgtcatcggatcagtcatgagcacggaataa

SEQ ID NO: 3 - DNA, 66 bp, Omphalotus olearius
  leader (1)..(66)
atgccctcgtcgctcttggacgctgctcgtgaatccggcgaagaggcatcccaaaacggtttccca

SEQ ID NO: 4 - DNA, 36 bp, Omphalotus olearius
  core (1)..(36)
tgggtcatcgtcgttggtgtcatcggtgtcatcgga

SEQ ID NO: 5 - DNA, 18 bp, Omphalotus olearius
  follower (1)..(18)
tcagtcatgagcacggaa

SEQ ID NO: 6 - DNA, 3905 bp, Omphalotus olearius
  ophP (1)..(3905): chromosomal DNA
atggaagaggatgtctttcttaacgacatcaatttgttgggggatttccatcacctgggggcagcggataagtaagtaacagtccgtgcagcatcgtctgtacgccactcccgctgtcagaatgtcaaacccttttctcgtgattacttgtttgagtacatactttctcctcctcgtcttagagttccagaataacggcaatcattagacgatgcatgcgaaagaccctgatctatcttagcaggtcagctgcacaatcgtgaatctacatatactcattgattcgtaccgatccgcgttggcattggaaatgttggggccaagtatggtactcagtctttcactctgcttccgttaatacgaatctcatcagacgtgatctgggtttcgtgtgctatgacggaacctttagtcttatcaccgaatggagtaacgagatcgcacgatgttcaggagaacacactgctcctcgagattgaagtggatacaatcgacgtcgctgatcttcttcttgtgctcagttgtaggacgatgtcgtttccaggatggggaccatatccacctgttgaaagggacgaaacttcagcaataacttacagtagcaagttgcatggctctgttacggttcgtgatccctacagtcaacttgaagtacctttcgaggatagtgaagaaacgaaggttcgtgaacgattccgcaattcttacaccttattctgaatggcacgaacaggcgtttgttcattcacaacgcaagtttgcacgaacctatctagatgaaaatcccgatcggtaggtatcctcgagttttctggcccactttgaccttactgcagtccttttacagagaagcatggctcgagacgctgaagaagagctggaattacaggagatttagcgcattgaaacctgaaagcgacggacattactacttcgagtataacgatggccttcaatctcagctttccctgtatcgtgtgaggatgggggaggaggatactgtcctcactgagtctggtcctggtggagaactctttttcaatcctaacttgctctcccttgatggcaacgccgcacttactggtttcgtcatgagtccatgtgggaattattgggcatatggtgtatctgaacatgttcgtatctatctcataatgcattcggacgttactcataggacgtcacagggatccgactggatgtctatttacgttcgaaagacgtcgtctccgcatcttccgtcacaagaacgaggaaaggatcctggacggatgaacgacaagattcgacacgtccgtttcttcattgtatcttggactagcgacagcaaaggtcaattctcaacgattgaagatgaggaattgaactagatggcgtttgcgttaggttttttctattctaggtatcctcctgaggatgacgaaggaaaaggtaatgcaccagcaatgaattgcatggtgagattcttttgatcgtagtttatcttgttcttgatgcgtctatcaggtctattatcatcggattggcgaggatcaggagagtgacgtccttgttcagtgagtactctatgtcgaatgatgccgatctcaaatgttgaagagtcttcagtgaggatcctgagcatccgttctggatctcgtcggtgcagctcactccgaggtaggctgtgtttcttcataaacgcgctatgctaattcgttgtttatgtagcggccgatacatcctctttgccgcgagcgtgcgtctggttcaattcacaccgctatcagttctctaaagatgcgtgcagcgcgacgcaagtcacactcaattagtcaagatcgcagatcttcacgaaaatgacattgggacgaatatgaaatggaaaaaccttcatgatccatgggaggctaggtttacaatgttcgtaagatctgaggttctaggtattcaattacttactaccatactggcagagtcggagacgagggttcaaagatctatttcatgaccaatctcaaggccaagaactacaaagtggcgacgtttgatgccaaccacccagacgagggtctgacgacgctcatagcagaagaccctaacgcgtttctcgtttcagcaagcattcacgcccaagacaagctcctacttgtttatcttcgtaatgctagccacgagattcatatccgtgatctaaccactgggaaacccctcgggcgtatctttgaggatttacttggacagtttatggtatcaggtcgacggcaggataacgatattttcgttctctttagcagtttcctctctcctggaactgtgtatcggtacgtagtagtcgtggaacccttctgttacccggactgatgcagagcccgaccaggtacacgttcggtgaagagaaaggacattcttcactatttcgtgctataagtattccgggcctcaatctagatgactttatgactgagtcggtgaggtcaattataccatcccatcgcgcttgcgactgaaataccatgatcaggttttctatccatccaaggacgggacttcgtaattactacaacttcacaaaaaatcacagaggactaacaaatcacgtagtgttcacatgtttatcacccgcccgaaggatgtactgctcgatggaacttcccctgtcctacaatacggctatggcggtttctccctagcaatgcttcctacattctccctctctacgctgctattctgcaaaatttacagggcgatctatgcgatacccaatattcggtgatccatcaagcgttcagattgctgtcctaatgctgacctactttagtggtggttcggaatacggcgagtcatggcaccgggaggtgagtctatgtcggtctgatgatcgatgatacatgttttgctgagcgttatccagggtatgttagacaagaagcagaacgtgtttgacgacttcaacgctgcaaccgaatggcttattgcgaataagtacgcaagcaaggatcgcattgccatccgaggaggatcaaatggaggtgcgaaacctattacctagctgaagttaattccgatctctgaatagtatccgatgttcgcaggtgttttgaccactgcatgtgcaaatcaagcaccgggactctaccgttgtgtgatcacaattgagggtataattgatatgcttagagtcagttgactacattctgaacttgctcattgtcttgctcactaattcctgtgtcaccagtttcctaagtttacatttggcgctagctggcgttctgaatatggcgatgtacgaatacctacacttactaagtatgcgatgattctgacggtcgcctaacggttgtagcctgaggatcctgaagatttcgacttcatattcaagttagtcaatctactatattgacttttcgtcactcaagataggactttctagatactccccctatcacaacatccctcccccgggagacactattatgccagccatgctattctttactgcggcgtatgatgatcgtgtctcgcctttgcacagtaggttcaacagaggttcgatggaatcgtatactttacttacccttacgcagcgttcaagcacgttgcggcattgcaacacaactttcccaaaggcccgaatccatgcctaatgcgcatcgacttgaattcgggacactttgctggcaaggtatgcattattggatggcggtatttacactatttgcttattatcgtccttgtagagtacgcaggagatgttggaagaaacggcagatgaatacaggtacgtccagcgacgaaatgtaagggctctctgtatttaactttcttagcttcatcgggaagtctatgggcctcactatgcagactcaaggttcagtagattcaagccgctggtcctgcgtaacagtttgaaaagcgtttga

SEQ ID NO: 7 - DNA, 2463 bp, Omphalotus oelarius
  ophP_cDNA (1)..(2463)
atgggccgtgaatctatactcattgattcgtaccgatccgcgttggcattggaaatgttggggccaaacgtgatctgggtttcgtgtgctatgacggaacctttagtcttatcaccgaatggagtaacgagatcgcacgatgttcaggagaacacactgctcctcgagattgaagtggatacaatcgacgtcgctgatcttcttcttgtgctcagttgtaggacgatgtcgtttccaggatggggaccatatccacctgttgaaagggacgaaacttcagcaataacttacagtagcaagttgcatggctctgttacggttcgtgatccctacagtcaacttgaagtacctttcgaggatagtgaagaaacgaaggcgtttgttcattcacaacgcaagtttgcacgaacctatctagatgaaaatcccgatcgagaagcatggctcgagacgctgaagaagagctggaattacaggagatttagcgcattgaaacctgaaagcgacggacattactacttcgagtataacgatggccttcaatctcagctttccctgtatcgtgtgaggatgggggaggaggatactgtcctcactgagtctggtcctggtggagaactctttttcaatcctaacttgctctcccttgatggcaacgccgcacttactggtttcgtcatgagtccatgtgggaattattgggcatatggtgtatctgaacatggatccgactggatgtctatttacgttcgaaagacgtcgtctccgcatcttccgtcacaagaacgaggaaaggatcctggacggatgaacgacaagattcgacacgtccgtttcttcattgtatcttggactagcgacagcaaaggttttttctattctaggtatcctcctgaggatgacgaaggaaaaggtaatgcaccagcaatgaattgcatggtctattatcatcggattggcgaggatcaggagagtgacgtccttgttcatgaggatcctgagcatccgttctggatctcgtcggtgcagctcactccgagcggccgatacatcctctttgccgcgagccgcgacgcaagtcacactcaattagtcaagatcgcagatcttcacgaaaatgacattgggacgaatatgaaatggaaaaaccttcatgatccatgggaggctaggtttacaatagtcggagacgagggttcaaagatctatttcatgaccaatctcaaggccaagaactacaaagtggcgacgtttgatgccaaccacccagacgagggtctgacgacgctcatagcagaagaccctaacgcgtttctcgtttcagcaagcattcacgcccaagacaagctcctacttgtttatcttcgtaatgctagccacgagattcatatccgtgatctaaccactgggaaacccctcgggcgtatctttgaggatttacttggacagtttatggtatcaggacgacggcaggataacgatattttcgttctctttagcagtttcctctctcctggaactgtgtatcggtacacgttcggtgaagagaaaggacattcttcactatttcgtgctataagtattccgggcctcaatctagatgactttatgactgagtcggttttctatccatccaaggacgggacttctgttcacatgtttatcacccgcccgaaggatgtactgctcgatggaacttcccctgtcctacaatacggctatggcggtttctccctagcaatgcttcctacattctccctctctacgctgctattctgcaaaatttacagggcgatctatgcgatacccaatattcgtggtggttcggaatacggcgagtcatggcaccgggagggtatgttagacaagaagcagaacgtgtttgacgacttcaacgctgcaaccgaatggcttattgcgaataagtacgcaagcaaggatcgcattgccatccgaggaggatcaaatggaggtgttttgaccactgcatgtgcaaatcaagcaccgggactctaccgttgtgtgatcacaattgagggtataattgatatgcttagatttcctaagtttacatttggcgctagctggcgttctgaatatggcgatcctgaggatcctgaagatttcgacttcatattcaaatactccccctatcacaacatccctcccccgggagacactattatgccagccatgctattctttactgcggcgtatgatgatcgtgtctcgcctttgcacacgttcaagcacgttgcggcattgcaacacaactttcccaaaggcccgaatccatgcctaatgcgcatcgacttgaattcgggacactttgctggcaagagtacgcaggagatgttggaagaaacggcagatgaatacagcttcatcgggaagtctatgggcctcactatgcagactcaaggttcagtagattcaagccgctggtcctgcgtaacagtttga

SEQ ID NO: 8 - DNA, 2066 bp, Omphalotus olearius
  ophM1 (1)..(2066): chromsomal DNA
atgtcatatccgctgacctctgacttaccgcccttcgtgaagtattatggaaaggctttcaaagttcctatgttagctggctggcaaattcttgtcacaggaccgcgtatgatggaggaaattcgaatggctcccgagctttctacccgtgaatccctcttcagagaggtacgctttcattttcaatactttgtgtgtggagcgttcgccaacgaatctctttagtgcctacagatggactataccttcggccctgaacatctcattaacccatatcattttcaatttgtacaaatctctctcggacgagatattggtcttcgaatacccgcgctacaagaggaaattgtaacgttattttcggaactcattcctcaaacgaaaggtgcgcagtcgtagttgaggctcgtgagcttccccgttatgtactagactattcacgaacattccctagattggattagactcccgggagcttctactgggcagcaaatcgcgttcagaacaacgactaggcttcttgttgggcttccactatgtacggaacgattcttatgcgcctctttatgtatcactgaggctaataccttcctcttgggccaggccgggaccaagactacaaggcactcaatgatcaatggcctgattctctggccaaggctgcgatcaccatcagcgttgttcctcagtttctgaaatcgtaagacgctcttataacgttgtagaactttcgatatgtatctgacctctcacgatttaggttcgtttctcgttctcttcccttttaccgatcagtgttagatcgcgcaatgaaccatgtgcgtcctttgttggaggagcgctttgaaaagcaaggaccattcggcgatgatgcaccggataaatcggtgcgtcttgtacccgtaggctcactgtcttactaacggaagtattcattcacgctcaggatgaccttcttacctggttaatgaagaaagcgccaacaaacgagaagtcactcgaagccattacgaggcgccttatggcaacacacttcgtgacttatcgtccgctatcactcgtatgtacttcataccacggaattcgttgaagaattgacctactattaactttcaacaggctacaacggaagtgctgtatgatctagcagcgcatccatcttacgtccctgagctacgagaagaagttgaagctctcgtgcgcgagcacggtgttacgaaggaaggtttggataaaatgtataagcttgatagcttcgttaaggaatgtctgcgcctccattcgagcccaaagagtaagtagatctcttaatttcattcacacgaattcgtctcaactcattgtttctctttcagtcaccatgtcacgtaaagttatgaaagacttcaccttttccgatgggaccaccattccagcaggaaatacgattgctgttgcaggctatgccattcatcacgatgaggtatgtcgtacatttatgcgacttgatatcgttttgacttttgggcaatagagaagctttcctaatcccgaggaattcgatgggttcagatattttaggaaacaggacgaagaacaggaaccgggaaaacatcagctggttactctcgctcacgactatgtcctctttggaggtggtagacacgcctggtaagtagtgaatcgtactgtatttattcctaggtggtgacataacattttacagtccgggacgctttttggtcgctgcgatactcaagacgatgctcgtgcatgtactatttacgtacgatgtgaagctggaggaagaaggtgtacgccccccagatgaatggcctgggatgttggttgcagtacctaaccaaactgcaggagttttggtgagaagacgagaattatcttcttgggaatgaataccgtgtgtctgtgtatgtgtatcctgtattatgaaggagaagaatcgcagtgtatttgctttcaatgagtaccctcgtataacgcatgagacggtcatcgacgttaagtacgttgtgatgcggagatatgtagcgccttcggaccagtcttgtgaaagttcagaagtttga

SEQ ID NO: 9 - DNA, 1864 bp, Omphalotus oelarius
  ophM2 (1)..(1864): chromosomal DNA
atggccgacgagtgggacgaattgttatcttggctcgtatcgagacttcaatttgacctctatgtccagtacagcgcgaatagagcacttggaagcttcgttctgctgctcggaatcgctatccttgtccttttcacgaagaaaatcaaagagatatacatcgacttccttgccattgcatatggttacgacttgatgagatcccgcttcaaatacggcgagaacgtttttcatttccatctctttggtgtacgtatctccagtcttacctttcgtcgtttatatactcagtgttgacagaatctcgtcatcggattacgaggtcagaaagcacggagagtgttgttcgaagagaagaacttcagtagtagtgatggatacaagatgttcagcaaagtggtccctgtatgtgaagctttctacttaccagttaccattgggaactttcgtgtagattcctgaggtcacttttgccccggaacaagaagattacaccttggcattcaagaagcacctagggactctgctggggaaggatagacttaatgacagtgagttaccgtatgacaccaccatgctccaacttgacttcttccgccagtaattgctaccatgtttcgagacatagaacgtgtcatggaaacttggggcgagaagggcgctatggatccgttcaataacatcaacgacgtatgtctcctctcctttcctttaccattctgtgaacttcacaaactcagatcgcgtttctgttgaccgtgcatatggttaccagcgacgaattcgtttcggatctctctgctctgtctcgatttcaaagccacttttccagacagctaagcagtcatacccccggaacgattttcttcccatggttttatcggacagctcagcgcgagagagagaagtccgtaatggccctcttcaatacattaaccgaacacatcgaggcacgaaaagggcgtaccgcaccgacacctgaagctgtcgatcttttactggcccgtggactgaatactcaagagattttccagtttatccttggattcttattcggaggtgcatttaatacaaccaaagcgagtatgttcgagctttattgtgatttttttctattcatggtcaatacatacagcgtcatggattcttctttatctcgcactccatcctcagtggggctctactattcgctccgaaatagagaccgttctctctcgcacatcaattgacaactctgaatcgatgtgcagcagactttcttcaatctctgtcgaagattgggaaagctcaatgcctacactcgaccttgtcatatctgaaactcttcgattggtcatgaatcaaaccgtgcttcggcgcaatatgggcgagaagttgaagatagatggtttaggcgaaataccgagaggcgcatttctggtctacagtaatgccgatgctcatttgaaccccgagctgtatcctgatccttggacattcaacccccagcacttcagtcagggtaaggaagatgatgagtatgcgttcttgggatggggcgcgggtgagcgttcatttgatcaaaaactttgattcacaattcacaatagtgcaatgcttcaacagggaggcaccggtgtgttggggttaagattgcaaaattggcaatcaaggcgattgtctcgtcattcataatgacttatgagcacgatgtcatcagctcaaatcgcaaagtcacctcgaaaactcccattcccgattataacaagataaacaatgtgagcaagttacaaacttggaatttctcgtgcgagactaattctggaatcagggacacccgaagaatggagtgtattccatcagctatagtaggcgcgtaggatga

SEQ ID NO: 10 - DNA, 1497 bp, Omphalotus olearius
  ophM2_cDNA (1)..(1497)
atggccgacgagtgggacgaattgttatcttggctcgtatcgagacttcaatttgacctctatgtccagtacagcgcgaatagagcacttggaagcttcgttctgctgctcggaatcgctatccttgtccttttcacgaagaaaatcaaagagatatacatcgacttccttgccattgcatatggttacgacttgatgagatcccgcttcaaatacggcgagaacgtttttcatttccatctctttggtaatctcgtcatcggattacgaggtcagaaagcacggagagtgttgttcgaagagaagaacttcagtagtagtgatggatacaagatgttcagcaaagtggtccctattcctgaggtcacttttgccccggaacaagaagattacaccttggcattcaagaagcacctagggactctgctggggaaggatagacttaatgacataattgctaccatgtttcgagacatagaacgtgtcatggaaacttggggcgagaagggcgctatggatccgttcaataacatcaacgacatcgcgtttctgttgaccgtgcatatggttaccagcgacgaattcgtttcggatctctctgctctgtctcgatttcaaagccacttttccagacagctaagcagtcatacccccggaacgattttcttcccatggttttatcggacagctcagcgcgagagagagaagtccgtaatggccctcttcaatacattaaccgaacacatcgaggcacgaaaagggcgtaccgcaccgacacctgaagctgtcgatcttttactggcccgtggactgaatactcaagagattttccagtttatccttggattcttattcggaggtgcatttaatacaaccaaagcgacgtcatggattcttctttatctcgcactccatcctcagtggggctctactattcgctccgaaatagagaccgttctctctcgcacatcaattgacaactctgaatcgatgtgcagcagactttcttcaatctctgtcgaagattgggaaagctcaatgcctacactcgaccttgtcatatctgaaactcttcgattggtcatgaatcaaaccgtgcttcggcgcaatatgggcgagaagttgaagatagatggtttaggcgaaataccgagaggcgcatttctggtctacagtaatgccgatgctcatttgaaccccgagctgtatcctgatccttggacattcaacccccagcacttcagtcagggtaaggaagatgatgagtatgcgttcttgggatggggcgcggggaggcaccggtgtgttggggttaagattgcaaaattggcaatcaaggcgattgtctcgtcattcataatgacttatgagcacgatgtcatcagctcaaatcgcaaagtcacctcgaaaactcccattcccgattataacaagataaacaatggacacccgaagaatggagtgtattccatcagctatagtaggcgcgtaggatga

SEQ ID NO: 11 - DNA, 1234 bp, Omphalotus olearius
  ophA (1)..(1234): chromosomal DNA
atgagttatagtcgaatccttgtccttgctgctgtgtccaaatgcatatcgacatttggaatcgtgaccccgccttccccgcaccgatggatgcttttcgtgcctatcgtcgtaatcaatgcttactgttacttcagcactttcaccgacgataccatgcgtaggaacgccgtcatagaaactttggttgcttcggatttcttggtcctcacggacgttcagcgcgaattccgtcaggttggacaacgggaacctatctcgaaactcgggctttgggctcgattacaatgggctctccggctactactctcccctcgaggtgtagggtggacgtatgaacccacatcagctcttccttctcgccacaaccttccgcggcgcacgttccttcggactcagctcattcttcttgtatgggctatcattggaaacgatcttgcctacattcttaccttcaaggatccaggcttcgccaaacatgcacgtccgtttagcgagcagcctctcatctggcgtttctgggcaactgccgtcttctttttcaaaatcagaaagtgtaccgaagtttattacttcttcgtagccttcgtacttgtcggggtcaacatgtcagaaccaagttcttgggtggaattttttggaagttggtcggatgcatatactgttcgtcgttggtgggggtacgatatctcacttttgacttggcgcgtttgatcgtcttgacgagattgtttctctgtagaagagtatggcatcaaagcctgcgcagggtctgtagacattttggcggagacgtatatcattctgactactcatttagatgtttgcttcgcacgggaagtacctagctcatcgtgtcttcggttttcgcccaggatgcccagcgtcttcgtacactcaactctatgtcgcctttttcttatcggggatcattcatgtgacgtcgtacgatccacgtccgatgtggtatttcatgtctcaggcagtcgcgatcacctttgaggaaatcgttctcaccatagcgaagaaagctggcatgaaaagctcgaagggtactcgtctgctcggttatgtttgggtgtactgcaacctggtgttcactttgggcccactcttcgattccatgaatgcagtcggacttggtttgggagggtacgatgggacaatcttgctaggtcgttatcgtgactcggagtggatgacggggttgctgtctcatgcgactgcaaattga

SEQ ID NO: 12 - DNA, 1044 bp, Omphalotus olearius
  ophA_cDNA (1)..(1044)
atgagttatagtcgaatccttgtccttgctgctgtgtccaaatgcatatcgacatttggaatcgtgaccccgccttccccgcaccgatggatgcttttcgtgcctatcgtcgtaatcaatgcttactgttacttcagcactttcaccgacgataccatgcgtaggaacgccgtcatagaaactttggttgcttcggatttcttggtcctcacggacgttcagcgcgaattccgtcaggttggacaacgggaacctatctcgaaactcgggctttgggctcgattacaatgggctctccggctactactctcccctcgaggtgtagggtggacgtatgaacccacatcagctcttccttctcgccacaaccttccgcggcgcacgttccttcggactcagctcattcttcttgtatgggctatcattggaaacgatcttgcctacattcttaccttcaaggatccaggcttcgccaaacatgcacgtccgtttagcgagcagcctctcatctggcgtttctgggcaactgccgtcttctttttcaaaatcagaaagtgtaccgaagtttattacttcttcgtagccttcgtacttgtcggggtcaacatgtcagaaccaagttcttgggtggaattttttggagaagagtatggcatcaaagcctgcgcagggtctgtagacattttggcggagacgtatatcattctgactactcatttagatgtttgcttcgcacgggaagtacctagctcatcgtgtcttcggttttcgcccaggatgcccagcgtcttcgcagtcgcgatcacctttgaggaaatcgttctcaccatagcgaagaaagctggcatgaaaagctcgaagggtactcgtctgctcggttatgtttgggtgtactgcaacctggtgttcactttgggcccactcttcgattccatgaatgcagtcggacttggtttgggagggtacgatgggacaatcttgctaggtcgttatcgtgactcggagtggatgacggggttgctgtctcatgcgactgcaaattga

SEQ ID NO: 13 - DNA, 35339 bp, Omphalotus olearius
  omphalotin cluster (1)..(35339)
gtcggagaacgtgaagtctttcaaaacctttcgaggcatacttgctacattccgctccgtcaattagtggaacaatattattcggtaaataatctctaggacaatggtactcacattttccaccccctctcaggcgcagggattccttcagaaaactatcaagcttgtacatcttattgatcccctccttcgtgataccttcttctttaactatcggctctacctctgctcgcagttccggcacatatgatggatatgtcgccaagtcgaatagcgcttgcgtgagtgccttgtatccatcagtccctgtcaaaattctgacttcgcatagatgtgcccaccatagacgcaggtcggtacgctgcaaacatcgttgccagtagcctgatagtaattgcccgaacggatttgtcgctctctggtgctttctccatcaaccaagtgagtagatcattctagaactcgcacacatgcttagatataaaggatcagggtttacacttggaagtctaataccaaccggctttcctggccaatcttttccgagcgttctctccttttcaagtcgttcttcgattactggagcaacgtgtttcattgcacgcttcaaagtcttaggaatataaaagtataaagctttggcgacgattctgaatagcatatagcgtgagtgtgacgagtgcggttaccccaagagacgtgtggaaacgcacggtttgaaaaggtcaggtgtgagaccaacgaacatggcggtttttacgagtgcgtccgtatacgtatcgttgatatcgttgtagtccggattatgacctataaattacttcataggcagagatcattccgataacagaggttgaacgcacacaatggaggtcctacgaaaagtctattggtgacccggagaaccatggacttgcccgtaagccatgcgggaacgctagcccaatctagtatgataagtaggataatttttcatccatttgagatcttcttaccgtcagtaggaggaactaattcagagaaagctgttatcacttcttcttgcatttcaggaaatcggccacctaggtcgcgcgtgagggaaaggtgcacaaaacgaaagtgataagggttctcgagtatatggggctccatcgtgtagtcggtttgcatgccctaataagaacgattcaaatgtgaattagagcaagaatgagaattgagtacctccttgatagaaccatataaagataactcatcgtcgctggctttccggacatcctcaaggagatcgttaccggtaaccacgaaagtcatcatgaacgcagtcgggattgtgaacaacttgccgtagaactaccattcgaggattgagtgggtatcattagatcatctttttcgcatacatactttgtcgtagccctcctgaatgaaatcgcgagtaccaagcagaagttgaaaagttccatacaaggtcccaatgacgcctgtcggtcctacacgtataacgtgtctatactagagagatgccttatcgtctgagaattgggaactagacttgaacgcaaacgaccgtaccttgaagctagttttcctctcatcgataaaacatcgagttatgatcaaagcaacgacggatacaccaaatgcaagcaatatggacgtggatatcattgctaggagtgccacctggatgctactgggtataacgcacttattcagtggcggaaggcaccctcaaggatgtttaatctccgacccatctgtacttgaaatttcggcgaacgccgagagaccaaatactgcaaagctcccgaccctggtccacaagagagacatctcgcttggaagcaggactccatcatgtttttcctgtcgtgcgtcgtgcaacacgacatacccggatcattattaaaaatgtgattcggtatgtctgatagtcttttccatttgtgacattgtcagtggagatctaggaatttagatctgcctggttttcttgaatttttgtattttcgtcggattttctttcaagctcagccgtcacagctttccaagtatcacggcgtcttttcgacttcagtaccttgagggttcctgcaacctggtggcccggaagcaactgagtcgactcgccgccaacctcacgaatatacacatacgatgttcttcaccttttcaacatcttacatcgctctggtcgtcgccgttcttgtcgttgcccgctcaatcgttaccttgcgtgctaaacacaaggtttcgtattcattcatgttctatcctcgagctaaattgggttgacataccccacagctcgatctgcccaccatcggaggggcaggactcgtccagtcttatgtcgctgcatacaaatttctcttcggatcccgcgatctcattcaagaaggttatgaaaaggtcgtctagcgattccatgtcatatccgctgacctctgacttaccgcccttcgtgaagtattatggaaaggctttcaaagttcctatgttagctggctggcaaattcttgtcacaggaccgcgtatgatggaggaaattcgaatggctcccgagctttctacccgtgaatccctcttcagagaggtacgctttcattttcaatactttgtgtgtggagcgttcgccaacgaatctctttagtgcctacagatggactataccttcggccctgaacatctcattaacccatatcattttcaatttgtacaaatctctctcggacgagatattggtcttcgaatacccgcgctacaagaggaaattgtaacgttattttcggaactcattcctcaaacgaaaggtgcgcagtcgtagttgaggctcgtgagcttccccgttatgtactagactattcacgaacattccctagattggattagactcccgggagcttctactgggcagcaaatcgcgttcagaacaacgactaggcttcttgttgggcttccactatgtacggaacgattcttatgcgcctctttatgtatcactgaggctaataccttcctcttgggccaggccgggaccaagactacaaggcactcaatgatcaatggcctgattctctggccaaggctgcgatcaccatcagcgttgttcctcagtttctgaaatcgtaagacgctcttataacgttgtagaactttcgatatgtatctgacctctcacgatttaggttcgtttctcgttctcttcccttttaccgatcagtgttagatcgcgcaatgaaccatgtgcgtcctttgttggaggagcgctttgaaaagcaaggaccattcggcgatgatgcaccggataaatcggtgcgtcttgtacccgtaggctcactgtcttactaacggaagtattcattcacgctcaggatgaccttcttacctggttaatgaagaaagcgccaacaaacgagaagtcactcgaagccattacgaggcgccttatggcaacacacttcgtgacttatcgtccgctatcactcgtatgtacttcataccacggaattcgttgaagaattgacctactattaactttcaacaggctacaacggaagtgctgtatgatctagcagcgcatccatcttacgtccctgagctacgagaagaagttgaagctctcgtgcgcgagcacggtgttacgaaggaaggtttggataaaatgtataagcttgatagcttcgttaaggaatgtctgcgcctccattcgagcccaaagagtaagtagatctcttaatttcattcacacgaattcgtctcaactcattgtttctctttcagtcaccatgtcacgtaaagttatgaaagacttcaccttttccgatgggaccaccattccagcaggaaatacgattgctgttgcaggctatgccattcatcacgatgaggtatgtcgtacatttatgcgacttgatatcgttttgacttttgggcaatagagaagctttcctaatcccgaggaattcgatgggttcagatattttaggaaacaggacgaagaacaggaaccgggaaaacatcagctggttactctcgctcacgactatgtcctctttggaggtggtagacacgcctggtaagtagtgaatcgtactgtatttattcctaggtggtgacataacattttacagtccgggacgctttttggtcgctgcgatactcaagacgatgctcgtgcatgtactatttacgtacgatgtgaagctggaggaagaaggtgtacgccccccagatgaatggcctgggatgttggttgcagtacctaaccaaactgcaggagttttggtgagaagacgagaattatcttcttgggaatgaataccgtgtgtctgtgtatgtgtatcctgtattatgaaggagaagaatcgcagtgtatttgctttcaatgagtaccctcgtataacgcatgagacggtcatcgacgttaagtacgttgtgatgcggagatatgtagcgccttcggaccagtcttgtgaaagttcagaagtttgaatatcaagtatatcactggcataaacataaaaggaagtgatgtccttttcccgctcagaccaacggaacaacattgtagcaatatgcatgtgctccttagccgtgactcagtacccaatcgaaaacattcacaacgcgatctgacatatggattgtaattgtacaaagtatacaacatgagggtttacgatttaaaacacgctcctacactgctccaccgactctcaaaacgactgaatcagatggaaaacgatttcacttggaagttgggatcagtctcgaactttgcatcgctgatctggtcgccattgaattcgtagaccgccttgaacggaacctccctgaagaacagacagtcagtaccgatactcatctgagctgcagatcatatagcgcactcgactttgagcttgaccaaggatttgccctgttgagacatagtcagacaggatctggaagtcgaaagataggtattgatacttaccacgactacaacctttccatctacttctttgatatcgctgatgacagtctatgtaagccgtcgcgatgacatgagctctagttatcaatcaagaaagggagaagaccacttgccttgtaatctaagaccttcagcagctcattgtgagcagtgagcgcatcagtcttgcggaactgcacagacgtgggtcccccaaggaaggacgccgccaacgaagtgtggcttgagctcaaatcgtcagccacatgagcgccagcatgcacatgcttgccgtcgttgagaaggtcaaagtagttacgaacaatttgaactttgtcagacatgatgtggttgagcttgtgtggtattaagcaacctgaagtcgggctgaaatggatttatagcagattttatagcagcatctacttttcatgtgacatggatgggctgtgagatattcgacggggggctgcaacgagataagaggacgtacagatggggtagccttgtcatttccaccggaactagacctatcaccctgtcctcaccgaattgtcggaatctccaaaatcacagaatattctaacatgaaacgtaacctgatgcctatactaagcgtgtcagaattgcaaggagccaattgaagaacattcaggctgtcgaaatatggtgccccgatgtattgaggttattctcgcaaaggccgtctttcactctagacatatatatgttatctaagattgaatacatgtgatggattctgacctggttctctgcgtagactccgctggtgccactccgactcgatgaaaatctttccagcaaacactcctgctcaagccttcttcaagccatgaggtgtttgttcagtggcaaagatacgaaatgagaaaaaaaatatttgcaaaatgtgagctctcgttcacagcctgcgccagctggcaccgggcgcgtaagtgacggtatgtgagaaagcaaatcgatcgatcacatgatcgtgttttcttgaaaatgcggactgcgcagtgcgatcaacatgccttttacttacagtgaccagtacacaaataagggtacaatagctgttcaaataggaatggaagttaaatagagaacggaatgttctgatagtttccctattcgacatacacgtaggattttaaaaccgcctaagtgacacctgataccacacccgacgagaggagagattgaaatcggagaacatgagccaattcaagtgtatcaggaccaagctaaatgttttagtttggaatgttatggaactgtcacaggaatactgagatgtaaaaaatacatacaactcgttacatcattacatcgactaccttcctgataaccacctgtcacgtaaaagtcccaatactgacctgcctgtatagtcattgaattcaacaacgttcatgatattaaaccacggccccaatataagaacgagggcgcaatgtagcccaacactctgtaacgggcaagagtatgttgataatgcttgacagcaagaatgcgacgtaaatctgacgggtgtatgaggacgcattgctgccgtgagggaaacaaaagacgggggtggcgaggaacttcccgcgtattgagaagatctagactcttatggttaggagcacgttcctttgagcaatggaataagttcttactagatcttactcttcgccgctgctggttgccacattctcccaaaagatatcgtcaggattgctaatcgaagatttacggatccggatgccccacttacagaatcgacgtatggcgtatgtatcaccccattttccaaagaattctggccatgaattgggtttcgacattccaattcccacaaatatcatcgagcaacatttgagcgcctgctcctggccccaaagctagataccgatttttgttgtccagcaagattgttggcaagcgactattggtacttgagctaggccagttcttctacttcaataaacttggaatggatattgcagactgtcgccccgcgaagtcacccaagtaatgcttagtatgatccgagacatcgttcgagtcaccgtatgcgaccttgatatcatcggcgccagcatgccctccgttcctgggccttcttcaagaactaaagaagatcatagtattagtaagtactcggcatcaaattgccaattacggtatatacttataacatgcgtgagaacttctgagaagcaaaattattccccggcggttctcacgaggatttactgtgttgataaccgactatcactaccggaacctgccgattggggaacaaaaccaattggatatctcactttgaccagttgtatagatggactacatactcgtgtacaacgtcgcggaagactacaagtttggagtgcaggttgaactccgtatgaacctttcaaacatgtttctcaagataaggtcaaattcttgaagcctagctgacattagttgtgcgcttgaatcgcaaaaagcaaccctcattctttggtttaccctgaattcgaacatccgacacagctccaggaacgacagtaatgataaatcaacttgaagcaaacctaccaagactaggatgtcgttatacatagtccggcatcggaaccgcctcagggttgaagacttcgtggtcatgaacagtgcgacaattgacttgatctccaacttggcaagatttgataccaaacaagcggtgttatgcgatgctgaattagcaagtaattgtagcaacaagctgccccgagtcctaaaaatgcggacgggttttcctctctgtcaggtctgaatcattccgcatcaaacgaatgaggacttgagagtataaggccggatcgagatgtgtgtctgcaagagtgtaagccagaaatttttaatcttcaaagaaagccttgcgagcctcttgcattgatggcagtgataggaagctatgaatacaatatttcagctgccccaaatctgccacacttacgttgatgcattgcgagcgctcagtcgtaatccggaggtgacgatcgtatgcgcggaatagaaagagcctgaggtctgttgtactttactcttgacgaggatggcaaagctgtgacaaggaccagagcctcaggactcggagtagtgtaagccacaccgttgaacttaccttctccgcatatctcacaccaaagacattacaggacatttattaagagacaccgtagtccgtatatctggtctttgagatgcaccaaatctctggcggagaaaagacctcccaggaaagggctaatacctttcttcctaccgccgatgattagacaccagtgaacattcctacgcaaaattcgactacaactcacgctgtcagaagatctcattctatcatgatttggagcttccttagcttccatctccacaggatgaaagatgaaagatgggttttgtattttgaagtcacgttcgttcttttgttggcatgcacgatggaaccagcactggcagcagctcaaagtcctttgcgaggttttgaaggtcagctccgtaaacagtttacatatcagtctcatccaatatgtacgaactacagagtatagttacaattttcagcttatcttacgacagtgaagttaatttttctcttacctcgtctgtattcaattgaaacgaaaagccgaaaacaagttgaactcaacgtcacgcgtcaacccgcgctcagcttatcaaatgtcctctcacgaagactctgacccactcctctcgccagaatctttatcattgatcaacactgattactgctgagcagacttgggtgtcgtgaaaaggattagtgcggcattgatgatagacgtcgataaacctccgatgttatcgttactagatgtccagctatctcaataggcggtagtggcaaaaaataggtgacataagtgtccctagccttgtacatcgcattacttgtaccgtcaactcttgaacttactgatccttacacaggtgaccatcggagctcaaatggtatgatgagaatagataccgactgccaatctgtagaatttgaagaaggtaagtaggtgaggaacgccgttggcatgatgattatcttcaatgcccatctgtgctaagcgcagattcgtatcgcaaaagaagggtactagtccttttgatacaagagcattctcattccgacgacatgaataattgacgcggaggaaatagcacccttctgatggatatttgaagacagtgaacaaggaaggaggatcactcactccgttcttcattttttatttttattttcctttgcggaggctggcgtgtgaagctcgaatgaaaaaggaccttcgtttctttgctccaagaccggactaacaggtctgcacatatctagaacaatcagttggaaaatcattttctcgagcaagacgtctgtctcggaaaacataccatgcgatattatgttctaagtactttcctaatgtgaagatggctacattcgtatgtgttcactgcgtttactgactttggacaaggccatggacacaaatcactgctgaaatataaaggagctgaaaatccaagatattcttcatcaatatcgctgatcatatcatggagacttccactcagaccaaagctggctcactcaccatcgtcggtaccggtatcgagagtatcggacaaatgacgcttcaggcgttgtcctacatcgaagccgccgcgaaggttttctactgcgtcatcgaccccgccactgaggcattcatcctcaccaagaacaagaactgcgttgacttgtatcagtattacgacaatggcaagtccagattgaacacttacacccaaatgtcagaggtaagcttcgtacaccctcaacagtcgcccacaccccgacgctgacacacgcatagctcatggtcagggaagtccgcaagggcctcgatgtcgtgggcgtcttctacggccacccaggagtgttcgtgaacccgtctcaccgagctctggctatcgccaagagtgaaggctaccgagcgaggatgcttccaggcgtgtctgcggaagattgtctcttcgcggacttgtgtatcgatccttcgaacccgggttgcctgacctacgaggcatcggatttcctgatcagggatcgcccggtcagcatccacagtcacttggtcctgttccaagtcggatgcgtcggtattgctgactttaacttcactggattcgacgtaagttctttcactccaatagctcaagatcttccgcttacctcctcacgaatggccagaacaacaaattcggcgttctcgtcgaccgtctcgagcaagaatacggcgccgagcaccctgtcgtccattacatcgcagctatgatgccacaccaagacccagtcaccgataagtacaccgtcgcgcagctccgtgagcccgagatcgcgaagcgtgttggcggtgtctcgactttctacattcctcccaaggccaggaaagcatcgaacttggacatcataaggcgcctagagctcttgcctgctgggcaagttcccgacaagaaagcgcgtatttacccggccaaccagtgggagcccgatgttcccgaagtcgaaccctacagaccatctgaccaggctgccatcgctcagttggctgaccacgctcctcctgagcaatatcaacctctcgctacttcgaaagccatgtctgatgttatgacgaagttggctttggatccaaaggcactcgccgactacaaagctgatcaccgcgcctttgctcaatctgtccccgacttgacgcctcaggagcgtgcggctttggagctcggtgattcgtgggctattcgttgcgcgatgaagaatatgccctcgtcgctcttggacgctgctcgtgaatccggcgaagaggcatcccaaaacggtttcccatgggtcatcgtcgttggtgtcatcggtgtcatcggatcagtcatgagcacggaataaacggtttggaatgtcatgatatcgccgtccttggtaaccaattctttcttctgttagccaatcaagaacttgtaggattaatgtggatgtcgtcagaaaatagttctatgtacaagtcaagtcggttatctgtgattaacaatgtatgaaatttgaacctcatcattacatgaaaacttgcctttcactctcggaaacaatgaacgaatgatcttcgttctctgagctgctgtgatttcaagcaatgtattgttagaacataaccggcaggaactatcaattgatcacttacttatgtaacaatgttgcgccggtagtaggtacatattcgttatcttgatctgtcaacattatacacaatcagtgctaaagaaaaagatcaaaagagaatagatcacattttacagattctaagacgagcttgtttcttcgatgctgcgagattcataccccgatgttccgtaccatatgcacacattacttcctgcgaacacagagaagaacattgaaaacacaaaactaccgactatttccagcatttgagccatccatcatccgtagctcgtattcaatttgcagtcgcatgagacagcaaccccgtcatccactccgagtcacgataacgacctagcaagattgtcccatcgtaccctcccaaaccaagtccgactgcattcatggaatcgaagagtgggcccaaagtgaacaccaggttgcagtacacccaaacataaccgagcagacgagtacccttcgagcttttcatgccagctttcttcgctatggtgagaacgatttcctcaaaggtgatcgcgactgcctgagacatgaaataccacatcggacgtggatcgtacgacgtcacatgaatgatccccgataagaaaaaggcgacatagagttgagtgtacgaagacgctgggcatcctgggcgaaaaccgaagacacgatgagctaggtacttcccgtgcgaagcaaacatctaaatgagtagtcagaatgatatacgtctccgccaaaatgtctacagaccctgcgcaggctttgatgccatactcttctacagagaaacaatctcgtcaagacgatcaaacgcgccaagtcaaaagtgagatatcgtacccccaccaacgacgaacagtatatgcatccgaccaacttccaaaaaattccacccaagaacttggttctgacatgttgaccccgacaagtacgaaggctacgaagaagtaataaacttcggtacactttctgattttgaaaaagaagacggcagttgcccagaaacgccagatgagaggctgctcgctaaacggacgtgcatgtttggcgaagcctggatccttgaaggtaagaatgtaggcaagatcgtttccaatgatagcccatacaagaagaatgagctgagtccgaaggaacgtgcgccgcggaaggttgtggcgagaaggaagagctgatgtgggttcatacgtccaccctacacctcgaggggagagtagtagccggagagcccattgtaatcgagcccaaagcccgagtttcgagataggttcccgttgtccaacctgacggaattcgcgctgaacgtccgtgaggaccaagaaatccgaagcaaccaaagtttctatgacggcgttcctacgcatggtatcgtcggtgaaagtgctgaagtaacagtaagcattgattacgacgataggcacgaaaagcatccatcggtgcggggaaggcggggtcacgattccaaatgtcgatatgcatttggacacagcagcaaggacaaggattcgactataactcataactcgtcgatgactagagacgcaacgacgaacgtagagcgctgaggtctactatggagctcctttagtgcttgagtaccatcaagatagtcatatgtttgactcacaccccgttttccagattctactggttccgcaacgtatcaatgtattaagcccgtgacagacggcatgttaaggatcaaggtcctgactcctgcaagccccaaacgttcaaacttccaagcgaagtgtcattcatcggcagaccctgggtcgtgttcgcatattggcaaagcaaagtcgatggaagaaattaaaaaattagcaagcaggtgacggtcaaaagaatggacgaaaagaatttttcatccgtgaattacttacataagtgatgtggttacataatcaacatttcagattggcgctgctctccttctgctctccttctgctcaccggaacctttgtcatcggcatccttaacatcgtcgaagcaatggccgacgagtgggacgaattgttatcttggctcgtatcgagacttcaatttgacctctatgtccagtacagcgcgaatagagcacttggaagcttcgttctgctgctcggaatcgctatccttgtccttttcacgaagaaaatcaaagagatatacatcgacttccttgccattgcatatggttacgacttgatgagatcccgcttcaaatacggcgagaacgtttttcatttccatctctttggtgtacgtatctccagtcttacctttcgtcgtttatatactcagtgttgacagaatctcgtcatcggattacgaggtcagaaagcacggagagtgttgttcgaagagaagaacttcagtagtagtgatggatacaagatgttcagcaaagtggtccctgtatgtgaagctttctacttaccagttaccattgggaactttcgtgtagattcctgaggtcacttttgccccggaacaagaagattacaccttggcattcaagaagcacctagggactctgctggggaaggatagacttaatgacagtgagttaccgtatgacaccaccatgctccaacttgacttcttccgccagtaattgctaccatgtttcgagacatagaacgtgtcatggaaacttggggcgagaagggcgctatggatccgttcaataacatcaacgacgtatgtctcctctcctttcctttaccattctgtgaacttcacaaactcagatcgcgtttctgttgaccgtgcatatggttaccagcgacgaattcgtttcggatctctctgctctgtctcgatttcaaagccacttttccagacagctaagcagtcatacccccggaacgattttcttcccatggttttatcggacagctcagcgcgagagagagaagtccgtaatggccctcttcaatacattaaccgaacacatcgaggcacgaaaagggcgtaccgcaccgacacctgaagctgtcgatcttttactggcccgtggactgaatactcaagagattttccagtttatccttggattcttattcggaggtgcatttaatacaaccaaagcgagtatgttcgagctttattgtgatttttttctattcatggtcaatacatacagcgtcatggattcttctttatctcgcactccatcctcagtggggctctactattcgctccgaaatagagaccgttctctctcgcacatcaattgacaactctgaatcgatgtgcagcagactttcttcaatctctgtcgaagattgggaaagctcaatgcctacactcgaccttgtcatatctgaaactcttcgattggtcatgaatcaaaccgtgcttcggcgcaatatgggcgagaagttgaagatagatggtttaggcgaaataccgagaggcgcatttctggtctacagtaatgccgatgctcatttgaaccccgagctgtatcctgatccttggacattcaacccccagcacttcagtcagggtaaggaagatgatgagtatgcgttcttgggatggggcgcgggtgagcgttcatttgatcaaaaactttgattcacaattcacaatagtgcaatgcttcaacagggaggcaccggtgtgttggggttaagattgcaaaattggcaatcaaggcgattgtctcgtcattcataatgacttatgagcacgatgtcatcagctcaaatcgcaaagtcacctcgaaaactcccattcccgattataacaagataaacaatgtgagcaagttacaaacttggaatttctcgtgcgagactaattctggaatcagggacacccgaagaatggagtgtattccatcagctatagtaggcgcgtaggatgaggacgacgtacatgacatgagatctagagtctgttttactcgatgaggaacaagccgactgaaacgttcctttgagtgtccgacattaccttcgtgaagagcaacagcggcggaaaaagcttagcttcgactatgttaaacatctgacagtataaatgaacgcggctatcggactctatcggagtggctaatcgcgatatgtctgaggaatgaggaatgattgtaagtacagtatgtatgtatatatatttggcaatctttaattatcacaactgtggcggaatttcggggttttgaaaattcaaagagtagcttatctatgtagcacgaccctgaaatcgtagttcgccggctagtacagtaagagctagtcaactacccggagtgcgtcaggtgcgtcctcgtgctgaatgccgggacttaagacggtaggtcgcagtaggtagcggcaggtacactaggaacatacatatctagccgaaatgcaccacagaatctgatggaagggaagcctgcgggatcctgtaacgagtcaaagacaacatacaactactgtcagatgtatatcacaagaacgatcgagggatggctgcagtcctgctgtagtccttatgtgcaagttatgaggcgaacaacacgcggtcccgcgagccagtgcagggttcaaggtcatgcatcatgactaggactgcgcataatacgtactgacaattaggctagtacttgtattgaatcctgcaggaattggagtacacgtttcgtagaccaaccaaccaattatggtgtccttttcagaatatcattcaaagtataaccctgttaccctgctagacttcatgtgttccgtggtttcggaggatctcccgatatcgttcaatcaacacagcaaactccttcatcacttcgattgtactgacgagtcgtcttcaaacgcttttcaaactgttacgcaggaccagcggcttgaatctactgaaccttgagtctgcatagtgaggcccatagacttcccgatgaagctaagaaagttaaatacagagagcccttacatttcgtcgctggacgtacctgtattcatctgccgtttcttccaacatctcctgcgtactctacaaggacgataataagcaaatagtgtaaataccgccatccaataatgcataccttgccagcaaagtgtcccgaattcaagtcgatgcgcattaggcatggattcgggcctttgggaaagttgtgttgcaatgccgcaacgtgcttgaacgctgcgtaagggtaagtaaagtatacgattccatcgaacctctgttgaacctactgtgcaaaggcgagacacgatcatcatacgccgcagtaaagaatagcatggctggcataatagtgtctcccgggggagggatgttgtgatagggggagtatctagaaagtcctatcttgagtgacgaaaagtcaatatagtagattgactaacttgaatatgaagtcgaaatcttcaggatcctcaggctacaaccgttaggcgaccgtcagaatcatcgcatacttagtaagtgtaggtattcgtacatcgccatattcagaacgccagctagcgccaaatgtaaacttaggaaactggtgacacaggaattagtgagcaagacaatgagcaagttcagaatgtagtcaactgactctaagcatatcaattataccctcaattgtgatcacacaacggtagagtcccggtgcttgatttgcacatgcagtggtcaaaacacctgcgaacatcggatactattcagagatcggaattaacttcagctaggtaataggtttcgcacctccatttgatcctcctcggatggcaatgcgatccttgcttgcgtacttattcgcaataagccattcggttgcagcgttgaagtcgtcaaacacgttctgcttcttgtctaacataccctggataacgctcagcaaaacatgtatcatcgatcatcagaccgacatagactcacctcccggtgccatgactcgccgtattccgaaccaccactaaagtaggtcagcattaggacagcaatctgaacgcttgatggatcaccgaatattgggtatcgcatagatcgccctgtaaattttgcagaatagcagcgtagagagggagaatgtaggaagcattgctagggagaaaccgccatagccgtattgtaggacaggggaagttccatcgagcagtacatccttcgggcgggtgataaacatgtgaacactacgtgatttgttagtcctctgtgattttttgtgaagttgtagtaattacgaagtcccgtccttggatggatagaaaacctgatcatggtatttcagtcgcaagcgcgatgggatggtataattgacctcaccgactcagtcataaagtcatctagattgaggcccggaatacttatagcacgaaatagtgaagaatgtcctttctcttcaccgaacgtgtacctggtcgggctctgcatcagtccgggtaacagaagggttccacgactactacgtaccgatacacagttccaggagagaggaaactgctaaagagaacgaaaatatcgttatcctgccgtcgacctgataccataaactgtccaagtaaatcctcaaagatacgcccgaggggtttcccagtggttagatcacggatatgaatctcgtggctagcattacgaagataaacaagtaggagcttgtcttgggcgtgaatgcttgctgaaacgagaaacgcgttagggtcttctgctatgagcgtcgtcagaccctcgtctgggtggttggcatcaaacgtcgccactttgtagttcttggccttgagattggtcatgaaatagatctttgaaccctcgtctccgactctgccagtatggtagtaagtaattgaatacctagaacctcagatcttacgaacattgtaaacctagcctcccatggatcatgaaggtttttccatttcatattcgtcccaatgtcattttcgtgaagatctgcgatcttgactaattgagtgtgacttgcgtcgcgctgcacgcatctttagagaactgatagcggtgtgaattgaaccagacgcacgctcgcggcaaagaggatgtatcggccgctacataaacaacgaattagcatagcgcgtttatgaagaaacacagcctacctcggagtgagctgcaccgacgagatccagaacggatgctcaggatcctcactgaagactcttcaacatttgagatcggcatcattcgacatagagtactcactgaacaaggacgtcactctcctgatcctcgccaatccgatgataatagacctgatagacgcatcaagaacaagataaactacgatcaaaagaatctcaccatgcaattcattgctggtgcattaccttttccttcgtcatcctcaggaggatacctagaatagaaaaaacctaacgcaaacgccatctagttcaattcctcatcttcaatcgttgagaattgacctttgctgtcgctagtccaagatacaatgaagaaacggacgtgtcgaatcttgtcgttcatccgtccaggatcctttcctcgttcttgtgacggaagatgcggagacgacgtctttcgaacgtaaatagacatccagtcggatccctgtgacgtcctatgagtaacgtccgaatgcattatgagatagatacgaacatgttcagatacaccatatgcccaataattcccacatggactcatgacgaaaccagtaagtgcggcgttgccatcaagggagagcaagttaggattgaaaaagagttctccaccaggaccagactcagtgaggacagtatcctcctcccccatcctcacacgatacagggaaagctgagattgaaggccatcgttatactcgaagtagtaatgtccgtcgctttcaggtttcaatgcgctaaatctcctgtaattccagctcttcttcagcgtctcgagccatgcttctctgtaaaaggactgcagtaaggtcaaagtgggccagaaaactcgaggatacctaccgatcgggattttcatctagataggttcgtgcaaacttgcgttgtgaatgaacaaacgcctgttcgtgccattcagaataaggtgtaagaattgcggaatcgttcacgaaccttcgtttcttcactatcctcgaaaggtacttcaagttgactgtagggatcacgaaccgtaacagagccatgcaacttgctactgtaagttattgctgaagtttcgtccctttcaacaggtggatatggtccccatcctggaaacgacatcgtcctacaactgagcacaagaagaagatcagcgacgtcgattgtatccacttcaatctcgaggagcagtgtgttctcctgaacatcgtgcgatctcgttactccattcggtgataagactaaaggttccgtcatagcacacgaaacccagatcacgtctgatgagattcgtattaacggaagcagagtgaaagactgagtaccatacttggccccaacatttccaatgccaacgcggatcggtacgaatcaatgagtatatgtagattcacgattgtgcagctgacctgctaagatagatcagggtctttcgcatgcatcgtctaatgattgccgttattctggaactctaagacgaggaggagaaagtatgtactcaaacaagtaatcacgagaaaagggtttgacattctgacagcgggagtggcgtacagacgatgctgcacggactgttacttacttatccgctgcccccaggtgatggaaatcccccaacaaattgatgtcgttaagaaagacatcctcttccatcgatctttccaggcatcctgtcgacaagtgaacgtactgaaagaagaaaatcatgagagcctgatataaatcacatcgctgagcttcaggacaagaagatgagatgtaggagataagaacgcaattcataccaacaacagcacagtcccaaagtagatttcaccgccggagttgaccggggtgtagacctgaaccggatttcgacaaatcaccgcaagttggcccggttatacaacgccatcgactttgattcacaagccagcgaatatctccacccgctgcagcatacctgatgtttgccttgcctgttgatgttcatgaagctattattcgcgagctctttgccagaaaggacaaggcgaccgtaaagtctttatcgcttgtatgctcctccctgacgcatatctgccaacggcacctcctctcgcatctcaccatcacctgtcctacagcacaacgcaaatacgaacggggccagcgctcgagtcacgcaacgcaggagctactccaatcatcacctcacctcatcccaatcattcggtcgttgtccatcatcgcctctagcactacttggagacagatcgatgtcgctctcttatacatcctcgaaacgctcacgaatctgaacgagtttacttggaaagcttcatcggtctcatcgcacccagctcgctgggcggagctaccccatctattgcaggacgcagtaaaggatgcaattcggcgtccttcgttgacctcgctagttctccatttcgctgacttgactgaactgttaaatacaacgatcatcgtgtcaccctctttaaggcaattaaggctacagggcactggctattcaaatctcaccttggaactgacccgtcccagcataataccttgcgacgatgcgggtgaaccaaatctgcaggaactttttgtcacgtcatgcaagctagagtggattgatgatgacgagatgttctttggctggatgaacactgtccaaccgcatctcagcctcacgcgacttcacaagctcgccgtgctccgctgcatacttaattcgacgcaagtagagctgccctgggggccgttgttatatcacgaatccgttcggtcgaagctggaggagttatggttcgatcttgggtccacgcgtggtcctccagctggtgagctagtctcttcgatccgtagctaggcgcattaggtcctcatgacgtttttggccttcaggtactgtatatctcgaccagctgccatccttacgacgacttgggctgaccgcagagctttcgggtatggctgccacctggaacttgttatctggaattgctcagtttttcgagggcgagaacaacgttgagattactcaccttgcccttgtcttcagcttctccactacttctctcgtactcaacccgctcagcatgccgtggtcgcgattggacgcagctgtcgcagcgttgccctacttgaaatgggttgggattcaggtcatcgattgtagatgccagccttgcatgatgatccctgaaaattcggaatttttgctgccaatgcttgcggagcagggagttttgcactggtcaacgagcgggttggagtttccagcacaaaagtagctgtcaggcacaccggcaattcgtaccggttttctttcagccgctgacttggtttcaggatcgtttttcacagaattcaaatagtataccagcaaaacatggattttgattcggcatctgtcactatacaaccagtcttccttctaaggctctcacatcttgtcccatctctagactctagtagtaatgatctccaaattttcaccttgaacgacaagttgttttatttcaaagtccgccacctgagctcgcgaggatttcacccttgcggcagcactaagcatcagtgattcctcaaatctgtttgctgttgctatgaaactgtctccgcctattttcatgttctattgatgaacaataatttaaaaaagatcatcatgatttctagagtacaatataaagtcagggtgaatacagtgtatggtaccagcaccaaccgctgttcattgtcagaaccatgagaaaccttgcgacatggtaaagaccccattcctttttaccttgtatgtttgctaattaattttaggctgtgcctctttctcctaatgcctttatcttgtttcggaactacatttttgaattcagttccaaacattcctgctggtcaagatacattatttaacagatcttatattggtttagacacagaagatattccgaatgatcttcccttgacaatgtgcaacatctcaaactgcattggttttggtggctatcagtcatcaggcagtagtttggatttctgggtgttcttcagtgaaatggagacaaaccttaaacttgcatgggggtatgtgatatttgaacttgacaatggaccattctcaccaaattgtaatcaagatggtactttgtattttctgaacgctgtctgccatcagtacaatgtgcacaaatgtaatgatgccattgtcattgtctcactatcaagtgtggtcaactgaacttgttcagatttagcaattggttcttttatgggacaccagaggcagattcacccaagtaaggcttaattcatatcatagaatttccagacttgatgctcacacacagctcaccatataacacaaccctcagcacacctttgctccatcttgaagcaatatcaaatactctggctgaagatttctatttgaattgggatattcaagcagtgccagacaacataccaatttatcttgcaatgagcagcatagaaacttcctttgaatatcaggcggatatatcaactgcaatgaatggtacttggatattttcctctggggacttctggaaacccaatagtaagccatatgatgggtgagtcatgagtccttgttcattatttttgaatgacattgatttaattcaaggtggtatcagatatcagtatactggtgataaccactgttactttggatattgaggttatgtctgacattctttatcatactagtaattatgagaataacagtgataccattgcacctattattcaatcagcaagtaattggttctattatggtactgaatttgcaaagtaggtacacaatctacatgcattattctgctagttgatcaaaagatatagtccaacaacatctagcccacaaaatcagacaatcaatgctctatctccaactgcttccaatggcacctctattgctgccaagaaaacatcaatagggccaatagttggaggagttgcaggtggtataattcttactatggttgccctatttgtggtttatagaagatacacaatgagacgcaagttgaggacaaccatgggttttgggcaggaaccactcagcatggtacaaattgagccttttcaacttccaccatctcaagttgaactccaaccaagcaagttgcaaaatatatctatgcaacaggtatgtttatttgtctggtcttttctcactacagtcttaattgctatcaggatgttgcaagggccattcaacagacaagtgctgttccagcaggaaatgatgctgaaatggaagaaataccacaggaaagggaagaagagcaggagttgttgcgtgagctgcagattttgcggcgactacaggagatacggggtataaatgatggtccaccaacttaccactctgatgtatctgtgtagatcattgcataggtactattaccagtctgacagatctgtgtagatcattgcaatacgtactgttcttgaaatactggtaaaaagtatattatatacagaatttctggacaatgagatcctatatgagttgtaaaatagcactgtatctactaacatgacccaattttcatttccaacgttcactttacttctcttttctaccaggaaaggatcagagcaaagcctagcacaccgacaataagaaggtgaatcactattactgtatacaatcagcatatgtgagcaaaggaagattttgaatgaggactacaggttttttgtaggttttttgtttactatttctataagaaaagtccgataatctagacaggtgatgactgagtaaagaacgtggtgaggaaaaaagaagggatagatgaagagaaagatgtatcgtacagatatgtcatatatataagtacctccaaagagaacagcgtccccacctgaatctaacctcccctgccgtttggtatcatattggtatggagaatatacgatgataacgaagagcggtcatacgtagaacgtcaaacccgcgaaatgtacagggtggacagcaatagtaagagtgcagtaaagtgaaatgaaatgagaggggggagagggggagagggagggggtggggggcaaggcaaaataaagtatgaaagcaacgtaagttcagcaaagcattcgaatctcaatggagaagaataagcacggtacagacaatccctgaatatgaatgaaaatgatgaggaggcgcaacgccatgtgtgtagtgttcggttgagttcagttcgaaatcggtaatctcgaaggattagacaatgaacacctcgggtatgaacgaattaaggaataagaatgcggacggagagaaattatagagcatggagttgaaaaggaaagggcaagcgtgttaacatccatcatactaataacacaaaaaaaacatcagctcaaactcacgcacaaatcacccgacaacaaaacacacctgagagttcttaggtttatcatacatccaagcagtcgcgttagggtctgggaacgagacgtgtcagtaacgacaacgccgaaggtgcgtaaaccaaaactcacttccatacgcctccttatgactctgtatgtaatccgccgtaggataccccgtgggattacacccaacgttcgtctgtcccttccgctgataaactctcacataatccacgagcatctcagcggggaaaatcatcgatgtcaggtcaatggtttgccaattttctagtcggagcgggataagatttgcagagaggagaggaagcgcgaacaggagaaagcaacagaattatcatcagtacctgaatgcacgaaacgctatttcaaagaacacatacgactaattcccaaattcagaacgatactcatcggctcctccggtatcaacctttgtcctacaccacttccattggttccttgatcagggccaacagaagcagcacccatacgagcggtaggctgaccgtcgacttgccaggttatgaatccgtccgagcgggagttggggtcggcccagtattcgaagcctgttgcgtgcgcatagaacgacgacgaaagcaacgaggagggaatagagagagaggagcaagcagaggaagagaaaatcagaatccattcaacgcatatgagactagaagcgacgtaccatacgtaacgaacttcttgccgctcccttggaacccatcgtctggcaccgagctcaacgcagaaacagcttgctgaaccgcacttccgtgatacgaattaatcctcgtcttgttcgtgtcccatatcttgaattcatcttccgtatcctagtatcgtagaaacatgagttgattgagcttacttcgacttgtaaaaaaaaaggaacaagaggaacatcgggccgtcgaaaaaagaaaatacgacacgctcgaacagtgaaaaggtcagaaagcactcacgttcaagaagacgtaatcatgcgagaatggcgcaaactgcgcagactgcgacacgacctgacccacattcgtcactttgtccttctccgcttcgaggacatcaatctccggtgcgcctcttccaagccacgtacccgtgttggaccggaacggccctgggtggtcgggcgtgtacgatgatgtggcagggcatgtgcatgcgctggacgggtggagatgcaggtgtgtgaagcaggacaagtggaaagaggataatggtataatgggaggaggtacagggggcaaatgagaagataaaggggaagaagagatatgaattagtacaagaacacgaattaaattggattataaaacacttacgacaacctctgacctggtaaccaactcaactcgtagttgtactttgacctcgattggtcagagtgtaaagccgctgcgggacctgtaccgtctttgagcgtttgattggggaaggtaccgacgtcgcatgagtcatagctgagaagaaagggacgtcagtgatatcttcagagaagatgatatgaagcgccgcaagaaagaaaagaaaagggaaaaggaggaaaaaatcaatgaatgagaaaggcacgatcccgtccaccactctttctaccgcctcctttcttcccactcttctactcctcataactccctccacctcccattctattaatattccatttcgctagcgtcacagtcccgcagcaattcagcaccaccaaaaataaccgcgaaaactccacccagagtctccatctataaatatccactatatctttgcgatgtatcgcactgctctctccctttcccttctctcctccacgaccacgagaattccgcctgcagcctcgttcaacgtacacggtgcaactgccgccactcatagcatccagcctgggtccagcgcaacatactcaactctccccgcgaatcccaaccacaaaaacaaaaccgtgagagaagcaaaagacaaccccgacagccaaatccacctcgaagaccatgtcaaggccccacccatgctcaacattgcgagaaagccagcgtctggactacagcctccatttcgccactgaagcacgccacttcgcatgacgttatcctcgtaggacgaggacgtaacggacatggcaaaggaaaccttgaggtctcgaatgtacgcaggaaaagcacgagaaaccgtagtcattccagaggccacgtcacctgagcaccaccaacatataacgccgagcaaaagaaaaaaaaaacaccacgaaaagattcaaaacaagaaaatccctgaactgaactcacgtataaggccacatcccatccgtcgtcgcaccataaccaggtctacccagattccccatcgtccacgcaccaggccaatacccctgcgtgttggcatctggaccaggaagaacgatggatacctcaatatatccacctgtgaagaaggacgtgagtgagggtagaagagacgagaaatgtagtagaggtatggaagagggacagtattggccaaaaagaaaaccttcaactcactcgaaaaacaaaacttattccagctctgcaacatcccactcctatacgtcaaattatgattactcccaacaccaaacggcgccgtcgatccttgcgttaacccactctgaaccgtattcgctgaatccatcgtgatcactaggtacccatcacgagtcgtcacctgcgatgggtcgtaccattctaaatcggcagtggaaccgtaccataggtcgacagcttcccagtaggcgtcgtcgccggggtagaatgttcgtcccggggtttcgaactcgtcggagaagacaaggtcgtaggtctcgccgtcgaagcctgtgcgggtgtgtacagaggtaggggtggcgagatcaatgagggtcggggagaaagggaagacgggtagaggggcttggcctgttgagtttatcctggaagagggtgttagtgggaggaaaaagagaggaacaggaaaagaagaggcgtactggatgtttccgtctatggcttcgttgaactgctggtcatggtagaaccttacgaccgggtaaccgacaaagagagcgattaagctagtcattgagaacgttgtgagggtactgaagaagaaaaggaagaaaatggcatacgccaatattataacaaccaacaagctgacattcgctatacccctccaaggaaacctcccaccacctgccaaccctcctctcgagaacacctgagtcaatcccatgagtacgcgaacccacacacgcatacgacccaacgaaaactcaccttcttaccagccttttcatcaatcgtaggatcatgcaacaaatccatctcatcgacttccccaccagcatctccatctccatcctcaagaggatcatacgcatagggtatcaaagtcttcggtctccccccacctgtccacttgtcatctttttccccatcccctgacccggccacaggtgtcggatgtgagataccgccagttccactgcccaaattcactgctaggtagccactaccgtgcccaccgtatattgatggatatttcgactcgtcagcggatagcgatacaatcgaggagcggttcgacgagctagcactgagcgtcgattcgcgacggaaatgcggtgtgggaggaagtagagcatggtctgcacctgctgagggtaatggtggtggtgaaggtggtgaaggcaaagaagtgttgacttgaggtgaagctgctgcaaggaggttcgagaacgtggaatgtcgcgatagagatgtaggagaagatgacggggacacgaaatctgattcatcagcaaaacgatagagaatcatcgagcctctggacggcgaaaacccttgtggttggtcgttgtatgcgcctggaattggtctcgtactcatacgtggggtacccatgagaggcatcgaactgactcttgatgcaggtcgagatgtcagtccgactgttcctactcctgctcgtgagcccatactcgacaaagtcgaacctgagttagacccggaacgagtcgacgaagattttcttccctccgagtctacatcaggctcttgtgagcctggtgtagcagagctttggcccaatggactccgtgtaaaggaagaggtcggacgtggttgcgaagggtgtagtactgcagctggtgatggtgaaggtgaaggtgaaggtgaggaagatagcagttgctgtgattgtccctccgtcaatttctccatgttgtaaaggatagtcctaaagaaatgtcaaaaagaagaacgaatttcgtgatttattactcactggaagcacgaaattcaacgaacgaggtcacgaacgaaagaaagtcgctcagggcctgatgcagtccctctcttaatccaattcctaattttccccagctacctgatatatatcaaggaagcgaagagcagagcgacaaggcggacagtgtatcgtatcaccgtcgtagcctatcgtatcttgctgattcgcctcagagctaggattccggtcagcgcccgtcgaagtcgtccacccacgtcgtccgtcgtcgtcgttgtcgtgcggaaaaacgcagcaaaaagcaaccgagcctcacccaatcaacacacttccctcttcttcgagctcgtgactgaaattaggaagggaaaagagaagaaaaagcacacaagtcttgggaaatgaattcacaaaccgggcatactaaaccgtgtaacaaaccgactttcaccggccggcatcaagggtacacggcagtcaccggcccggggagccaagaggaaagaaattagtttacataatgaaaattttaaatttcatttcaactttcactacatacactatcaataatcagcaagtttcaagggaagtgcaggctaccaaagaatacttcactgctgctgaacgactcagattggccactttacagtcaagatagaggatgacgaaggaaccggcactcagtgtacagcgatattcggcgcggctgttcaggcatccaactttcaatccctcaacgtcaacgtcaacgtcaacgtccacatcaattatcgggaaagagctttgaaatgatatctctcaagttgaattgcacaagaacttgcgtacttgccggaggttggcgttgttgtcgagctcattcttgagtgaactggacgacgttgtatcttgtaagtcatctttgcatatctacactacctattaacatatctaaacgctccagatacttatcctgatttatgaacgcacatatgcgagttcagcttcagtactcagtgctcagccctcagccctcagccctcgcaactgtcctggtcaaagctaaacgcggacaaacttccggaatcatgggtacaacgtagctcccggtggactcgacactgctatccacggttgtttccaatgtaagttcgcccagaacctgcacccacactactggttactcaacaccacttcagggcaggcttcgcaaacctaatacatctcagcaaaccttattcaatcgctctgatttaagtagtgtggcgcgcgctattctcccttcttttcttccacaccccatgtcaaattccaaccaaaaaagtcccacaagcagctcgaaagaatcctccacgccttgcagattcttcctcaaaggacactgtaaattcggagagagctgcaatcgaagtcacgatgtgaaacctaggcctgctgtccaacgtcgaagttggatgcaatcattggctagcaatggggggcgtttttcggtgagctatcgtgtatctatcaagaaagattataactgaagtgctctcttttattgctggccccattagatcaattccccatcgacatcgcaacaaaggcacgacgttactccggagccttctgcaggtattactttcggcaacttgactttcacgaatgaagacctttcagaggcaagttatcgtcaaactactcaagtaattctcctctcatcaacccattagaacgactcctcatcggcatcggcactaccatcggagaaccacgtgcacgtctcaccaccgtctattgcggtgaacacctcaacacaacccacacgcccccagaaccaaaaacaaaacaaaccttgtttcgcatggagagccgggacgtgtcgtagaggggatacctgtaaattcaaacacgatccagaggtgaggggccgcaacccttccagcgcctttttttcgagtcattctggcttctggcgctaatatacgatcacatcatcctcatctgaattaggatcaggtgcagcagcagcgtgtatcctcacctgtcactgattcacctgttctcgcggcacacgagaatggcgaaacacgcggacagaccggcgctcgacgtcaacgtgcggataattggagggctcagcgtggagcgaaaggaacaaagaatgctcaggatccaaataattctacgggtactgctaatcaacagggctccgcgggaggaattaggggcccacaggattccccgagaagcgggggtggcggtggatctgttcctcgggaagaacagctccgaattcagagagaagctcgacgtgctgtgcaagaagcagaggaggaggaaaggcaacgtaaaactgccgaagaaattgctgcagaactcgatcgacaagtggagatgtggagacaacgtagagcagctgaggcggaacagctacgtattcagcgcgaagctgagcatgctgagagggaagcacagagaaggaaatcggaggaggaacgtgagcagcttcgtattcagcaggaagctcgtcgagctgagcgtgctcgaagggaagaagcattcactatccgacaagtcgagcttggcagcaccatcgtcacgttcagtgccggcgtagctatcgatcacatcgttactgggttcgagtcttgtcgcgtgcaagtgaagaatctcccatttgatgcgaaacgtgatgaagtctacgaaatgttccgagcgcagggtgttgaggaggggaaatttcatcttgtgaatatgaaaagggtgaaccagcgacaggaggcgagtttcattgtcgatggggaagttgggaaggcaatcgctcttgggcttgatgggacagatttccgtggtaatgccctcgatttcagtgtcagcgacagcggaactatcggcgggatgtattctacgggggacagtgctgtcctgaccgttcattggagagctcctagcgcgcgcttcgtagcgagctatgcatatgctgaaagtgccgatgcgaagatgcgagaggtgaatgagaaggtgatcaatggtcgaaggatcagagttgagcgaaatcggacgcgtcaagggtcactcgatccatgtgcactcttaatcagtggtttcacaccgcaaacatcggacgaggctgtgagggaagcctttcaggctctttctgtacgaaggctcccccatgctattgaatacgatgagaattacgcgaaaacctacgtcaggaatctcattaccgtaatctcaaatgtacggtttgaagggccaacactcaattctttggaaggcaattacaccttacgtgctcacttcgatacttgggaccaaaccaagttggctcgcgatcagtttcatcaggggaggtatccccagatcggaaatggtacctttcgactacggcttcccgatcccatccagtacaaaattaccattccttctcaacagcataaagcacagaagaaacaatgggacactttcgtctcagatactgcgggccaaaaaggaagctatgttctcatcacccagcaaaacgattccgtccggacttttgttcgcgtagggggagatgacagtcaagcagtcggtatgctcaaggttcgtgtcgagcgcttggctgcgggcgaaaagctagatttatggcatcgttcgtttgcgaatgcagccggacagagatttctcgagtctatataccctgctgtgggagctctcgtcatgcatgactggagaatgaagattgtcaaagtatacggggaaccttcagcgattgcgaaggctcgagagatgattatggaggagatagaaaggctttctggacttgaatatactgttgcgctgaagagacagtctatccggtttttcactcagcgtggaatcgcttcgctcaaagaaacctttggagaagatagcgttacgctcgacatctctacctctccttgtcgtatcaccattagaggcggggaagaggctcgtcatgctctcagccgtctcatagatgaatctctagacgaacttgcggttgctaatgctgcacccaaggacgaaagctccacatgccctatctgcttagacgaagtctcaacgccagtacaacttggatgcggacatgaatactgcactgcttgtatacgtcacttcttcactgctgacattaagacgttcccacttgtctgcatggggaacgaggcaacatgcttgaaacccatcgctttacccaccattcaacgattcctcacaccacagcagttcaacgttctcctcgaaaccgcctttgtgagtcacatcgagacgaacccgcaaagttacaagtactgtccgacacctgattgcgtgcaaatttatcgccgcaccacaaacgataccgctgctcttcagctgcgttgcccgtcctgtctcattacgatctgctccagatgccaagaaggccatgatgggatgacttgtgaagagaggagaatccagggcgatccagcagaacaggaacgtctgaatgaagagtgggcgcgaacggcaggggtgaagaaatgtccttcttgcaacgtctggattgaaaagaccgagggatgcaatcacatgtcgtgcaaatgtggtgctcatgtgtgttgggtttgtttgggtatattcgaggcagggactatctatgaacatatgggaagggctcatggagggatctttgaagacaataatcgcccgagggtagtgcagcagcctgaacgggtgattgaccgagatgacgatttcgattttctcgaacaagcacgccggctacgggttctcgaggctaataggttgcgccgacaggaggatgtgcgagtgcagagggaatatatcctagcggatgaagtacgaaggaggactcaagctcaggctcaaggaatccgagcattgagagaggctgaacggatgatagaggatggggaacagagaaggagggctcaagagtggcaacgacaacagcaagcggcgttcgcccaagagcgtagattgcaactgctgagagagcaggaacagcgcgagcgtgaaagtaggcgagatggaggtggttggggttgtactgttatgtgagatgtgtttgtatgtaagatatccgatgatcgatgatccgtaatggtcatttgttttgaacgtgcatcactcagagacttatcatcgactctcctgatgaagctaagtgtattaaacatacaagaactctcactcgtacaccaagcggcttgagaccgaaatcagtcgagctatcaaggaatcagtgtatccgagagatcagtccttaataaaccttggtctcttccgtgtacccgccaactacactcacgaacggcgaaccgtgagtgaaaatgtaattgattttgttggacaagtacctggtgcgacataaaacatcgtataaggtcccgtgtagtatagatctcgaccttctaggcagctttgttcctggccattggcccatgcagtattcgtgaacccggttgactgacataaccccaaggtatcattcacggtgggattcaatgcctttagtggtactatggtagaatatcagtgagcttttcagcagaagaggtatcgaaattgattctttgaaggttgactctgaagcatgcacgcatgccacataagtcatttctgctcttgatgttccaagcacacatacctacctacggtccgagtccactttaacatgtactgtttcttgtgataatgtccttgacaaatggatctatgcaggtatacaattccttccctcccccgtcaagatggcatcgtgtgtctccgcgatcgccgttatatatgtaggtcggcatacagcggaagttcgaaaagagtacgcatataaaggtgtatttcaagtacgaagctatccaggcaaactccagccacttcttcctaaccttcgtataaaggtgaaaagcctgttctctgtctcgaactgaaaactcactcgttatcattcatccagtttgattatcatgatgttcggtggtcgcaacctcactcttcttgtcgctgctgtgttcgcggtcttgatccaaggaggtaagctaaatcctgcacaagttctcgattagatgccaacatctgatatcgtcgtgaatgtagctgttgctagccctgtaggacctgcggaagctgcgctcgcagcccgcaatgaggaatgcggtgcagtttacgagggcgagctctgcgtaagctgctgtggaggtccctaggtctatcacaaataagtactgtcatgagttatccaaacgtattctgattcatcacgtacatggaaaaagcatttcaatgaactcggtggaggcattcctgcttcttggctactagaagaatgcgctaagatgagctacgactgaatgattgagccgtatacacaacttcaacagaggttttccgagtcgacgcgaggctgtccctgaagtggccctactccctctccatagctaacgtttcgcgtgttattgcactcgcacttgtagttacgagtgcagggaacggagtgaccgg

SEQ ID NO: 14 - DNA, 43 bp, Omphalotus olearius
  ophMA_fw (1)..(43): primer
cgtcgacgagaacttgtatttccaggagacttccactcagacc

SEQ ID NO: 15 - DNA, 33 bp, Omphalotus olearius
  ophMA_rev (1)..(33): primer
gcggccgcattattccgtgctcatgactgatcc

SEQ ID NO: 16 - DNA, 42 bp, Omphalotus olearius
  ophP_fw (1)..(42): primer
ctaattattcgaaggatcctacatgagcgataaaattattca

SEQ ID NO: 17 - DNA, 40 bp, Omphalotus olearius
  SalI_rev (1)..(40): primer
agaacgaaaatatcgttatcctgccgtcgtcctgatacca

SEQ ID NO: 18 - DNA, 40 bp, Omphalotus olearius
  SalI_fw (1)..(40): primer
tggtatcaggacgacggcaggataacgatattttcgttct

SEQ ID NO: 19 - DNA, 41 bp, Omphalotus olearius
  ophP_rev (1)..(41): primer
ggccgccctagggaattctactcaaactgttacgcaggacc

SEQ ID NO: 20 - DNA, 1367 bp, Lentinula edodes
  methylase (1)..(1367): chromosomal DNA
atggagactcctaccttaaacaaatccggctccctcactatcgttggcactggcatcgagagtattggtcaaatgacccttcagaccttgtcttacatcgaagccgccgacaaagttttttactgtgtcatagaccctgccaccgaagcattcatcctcaccaagaacaaggactgtgtcgacctgtaccagtactacgataatggcaagtccaggatggatacctacacccaaatgtctgaggtgagatgtttcttgtcaaatgttttctttgtgttctgtatagagctgacatcttcaaaggttatgctcagggaggtccgcaagggccttgatgttgtaggcgtcttctacggacacccaggtgtgttcgttaacccttcgcttcgagcgctcgctatcgccaaaagtgaggggttcaaggctaggatgctgccgggcgtatctgcggaggactgcctttatgcagacttgtgcatcgatccttcgaaccccggatgtctgacctacgaagcatctgacttcctcattcgagagaggccaacgaacatttacagccattttatccttttccaagttggatgtgtcggtattgctgacttcaacttcactggatttgaagtaagtatcatcaatctccatgaggtatacatttaataaactctttgttctagaactcgaaattcggaattcttgttgaccgcctcgagaaggagtatggcgcagaacatcctgtcgtgcactacatcgccgctatgctccctcacgaggatcccgttaccgatcagtggaccattggacaactccgtgagccggagttctacaaacgcgtcggcggtgtttccactttctacatcccgccaaaggagcgaaaggagattaacgttgatatcattcgtgaactcaagtttctccctgaggggaaagttccggatacgcgtactcagatttacccacccaaccaatgggaacccgaggtaccgactgttcctgcgtatggatcaaatgagcacgccgctatcgctcagttggacactcacactcctcccgagcaatatcaacctctcgctacttccaaagccatgactgatgtgatgaccaagttggcgttggatcccaaggcgctcgcagaatacaaggctgaccaccgagccttcgctcagtccgttccggatttgacggcgaatgagaggaccgctttggagattggagactcctgggcgttccgctgcgctatgaaggaaatgcctatttcactcctagacaacgccaagcagtcaatggaagaagcctccgaacaaggcttcccgtggatcatcgtcgttggtgtcgttggtgtcgttggttctgttgttagtagcgcctga

SEQ ID NO: 21 - DNA, 3296 bp, Lentinula edodes
  oligopeptidase (1)..(3296): chromosomal DNA
atgttttccgcaacccaggagagtcccactatgtcagttccgcagtgggatccatacccaccagtcagtagagacgaaacctcagcaatcacgtatcagagcaagttgtgcggttctgttactgtacgcgatccgtacagtgcacttgaggtgccttttgatgatagtgaagagaccaaagtttgtggaaatcatgattccgtgtacaggatcctcaatgatcaggatataggcctttgttcatgcacaacgcaagtttgcgcggacgtatctagacgagattcctgacaggtatatgtctaatgtgctctttatcagtcactaacgcttttatttgacagagaaacgtggctgcaaacgctgaaagagagttggaactacagacgattcactgtccccaaacgcgaaagcgatggttacacctacttcgaatacaacgacggtcttcaatctcaaatgtctctacgtcgcgtgaaagtgagcgaagaggacaccattctcaccgagtctggacctggtggggaactcttctttgatcctaacctgctctctctggatggaaacgcggccctaactggctcaatgatgagtccatgtggcaaatactgggcttacggcgtttctgaacatgttcgtgtgttgtcataagggagcactttcaagttttactgatacaacatgagaaagggttctgattggatgacaacatatgttcggaaaacatcgtcaccgcatatgccttctcaagagaagggcaaagatcctggaagaatggacgacgttatccgttactctcgcttctttattgtatattggtcgagtgacagcaaaggtttggtcgcaatgtgcaaatggttatcaaagaggcctacttattcttaacggtctaggttttttttattctagatatccaccagaagatgacgagggcaaaggcaatacacctgcgcagaattgcatggtgagtttctttccacgctctatgtttactactctcatttccaacgccatcaggtgtattatcaccgcctcggggagaaacaggaaaaagatactcttgtctagtaagttcatgaggcatcaacacatggccttgcttatgaatattctgaaagtgaggatcccgagcatccattctggctctgggcactgcagctctcccctaggttagtgtttcccagcactcctgatcactgtattcacttagcaaatatgtagtggccgatatgcactcttgactgcaagcgtacgcaaaactcatactatatacacatctcactctaatgtttagctacacatagcgggatgctagtcatactcaactagccaaaattgctgatattgggacgagcgacattcaaaacggtattcaatggttgactatccatgatcaatggcaggctagatttgttatgtacgtctaccttctcttctgcttctcgacctcgactaacattctagttaatactgatctcattagaattggagacgatgattcaacaatttatttcatgacgaacctagaagctaaaaattacctagtagcaaccttggacattcggcattcagaagcgggggtcaagacactggtggctgaaaatcctgacgcccttctcatatcggcaagcatcctctcgaccgataaactcgtcctcgtctaccttcataatgccaggcatgaaatccatgtacacgatcttaacaccggaaaaccgatccgccagatattcgacaatttgatcgggcagttttcgctgtcgggacggcgtgacgataacgatatgtttgtctttcacagtggtttcacttctcccggcaccatatatcggtgagctcgttgataagacattgtggcaccctcgccgacattgtacaggttccgattgaatgaagatagtaacaagggcaccctatttcgtgctgtacaggttcctggcctcaacctgagtgatttcacgacagaatcggtatgcatacgtgcttctcttcatcgctcctattgctaagcaggatatgcaggtgttttatccatcaaaggatgggactccgtaatcattcttgctgaatctcttaaacatccgaagcctgataataaatttagtattcacatgttcattactcgattgaaggatacccccgtcgacggaactgcccctgtatatatttatggctacggcggcttcgccctggcaatgctcccgaccttctctgtctcgacgcttctgttctgcaagatctaccgggcaatgtatgtggtacccaacatacggtaattaattcatcatcgtttccctttcggtctctgaattggcttatgcttcagtggtggttcagagtttggagaatcatggcaccgggaggtgagtagtgtttaatttttgcgtaaaaaataactttgtctcaggcgcgttctacagggaatgttggacaagaaacagaacgtgttcgacgacttcaatgcagctacgaaatggctcgttgcgaacaaatatgccaataaatataacgttgccattcgcggaggatccaacggaggtgagctgagctgtaactgttcgaacctttgatcacagacattaagtgatgtggtttttctacaggagttttgacaactgcgtgtgcgaatcaagcgcctgagctctaccgctgtgtcatcaccattggaggtatcatcgacatgctcagagtttgtcaccatatccttcgttctgattcctcctaattcctcactcatccatgacattgcagtttcccaagtttactttcggcgctttatggcgttcggaatacggtgatgtactaaaatgtcaattggtctgaaaaaatctgaccctttatagcccgaggatccggaggattttgacttcatttacaagtgagctgaacagtcccgatacaagattgcttccttacccaaatcttgacaggtattccccttatcataacattccatcaggagatgtagtcttgccagctatgcttttctttactgctgcgtatgatgaccgagtttctcctctgcactgtgagtgggccaaaatcttgaggtcttatagaactctgatcggtttaaagcattcaaacatgttgctgccttacagtataacttccccaacggtccaaacccggttttgatgcgtattgatctaaacactggacatttcgccggcaaggtttgaaattttttactgtggtctcgcactttgctaattccatcccagagtacacagaaaatgctcgaagaaaccgccgacgagtacaggtgcgacttactctgttgtaacctacaattgtga

SEQ ID NO: 22 - DNA, 25900 bp, Lentinula edodes
  cluster biosynthesis (1)..(25900): chromosomal DNA
ataattaatcataataaggcttgagttatgaatacatgagcaataaatgtacgtacagcgagatgcaaggagaaacgaagtttcgaaatatacattcatgtgtttcaactaagccaagttatatgtattgtatgcacagcagatcgaaagcaagaaaagaatcatctaccgctggagaaggagcccagtctcagctagagatgcctgcatctagtagcgacaaccccgtgagctgactcactcacaatatgattaatcgtagatgacaggctctcaggcagtagagagaaaagcataaaggaaagaaaccagctacaactggattaattcttcatctatacaaagtcccgtccacttcgttcccgtcatgatcaaaactgaacgaccttaaaaacagattgtgagaggcccttggcgtgggtgcagcgaaagccgggataggctcaggctcataaccgcggccgtcatcgtagtcgccaacttcgttcaagtctaagcctagggcgttgacatctccgggaggaggactatgaggagtctgggagttagagtcacgacttgcacggctagggccgctgacagagccaccgttaggcgccacatgtgtcacaggtgccataggaattgcaggtgccgagttgtattctggaaaggctggacgatggggagcactagccagtgcggaagaaggataaccgaaattaggtagtgtaggtagggggttggaattgagattggaggttgggatagggcgcaaagactgaggacgatgaccacgagcatgatcatagttgcgggtgttgtcgcgaggcgtcgtgttgacagtgttgacaggaagggaggtgtatcgctgatttactggtctagtattccccgtcccagcagattgactgacttgactgacattaccacgaggagcagcgcgggaggcagtcggtgtttcggagtcatgatgcgtgattgcgtggttgttcgtttgtacgtcggatctagcagaagaactaacgacaggggctgagaaacccgccacttcatttgtcccaactgcagatggtcgattcgggttgtttgcatttacaggaataacaggcaggaatgctctgggatcacccaccggtatctcctgagaggatgcaagactcgcgcgactcgaatgatgcgcatgctcaggaaacccgcgcagattagccatcacgtcacttgcattggattgtcttcgggatgaattagcggtcgttactgtaccccaagtgggcgtctcgctagggacagcggaagctcggttgttagcaaaatgttgggcttgggcttgggatgatgaacgaagggggtcttccatcaagtcgcggagccgattggcgaggctttctggggtgatgctttgtgttgtgggctgtgttgtcgtcgtgcctcgtctcaatgcctgctggtctccattggaagtggttgcagaggcggtagcaggaggtattgcggattcagtcgcgccattgaatctcactgaccgaggaagtggagcagagactggggggccaggagttgttcttgctctcgcgctgaatccactgttcaatccctccgtgtacccgctggcaaaagccgcttggggatcaactgcacgcctctcctcgaaggactcatctatcaaccctaaaccagtgacaccagctatatcttctcttccaccactcagatgctcttctctacgacgcgttctcgattttgacttgtgtcgacgacggtgtttgcgggataagtcctctactggggcactggctggtggaggaatggaagatgaggcatacccattgacgtatgtcgtctgtgagccggcttgagaccagtgttggctggcaccgggcggaacatagattctgtcattttcggcagcgcctggaacgattctgtgtcttctagacggcgaagagcgcgatgcatattgttgcgcctgagaaacggcagcgtcaagtacgacgggaagtgcttctagtcgagaagatcgacgcggacgagatgttagtggagggatgggtagagggtaatttggaatgggtacgtagttagcaggaggctccatgtaatgttgagaagacacaacgacttcgggagtgtgtggcatagcactggcaaggccgggggcgacaagaccgttggtgtctcgtggacgggaagagcctgcgccagctgatgaagttgcaaggtcggatgaggacccatcccagcctcgacgatcatgtatagatgcctctattgctgcagcgacctgttcagcttcgacagacgaactacgtatatcccctgcgactgtggcgtatctttccatagtaatagccatagggggggctggcagcggattgtgacgtagagaatcgttctctgaagtgtaatccgtgacccttgctttgcctttccctttgtctcgtctactaaattctagacgggcaagttgattgcgcagactttggacttctgattgggattgttggagagttctgtcatgatagtcagaccaataagcatggctgactcggggaggacttacgtttcaagttcctcgatggtagtagagaaactggcctccattgcgagtcccatatcacgatcgtgcacgttgctgcgttgaagatgcattaaagaagtttccagtcgcttgatcctgaatctatcttctgcttttcgttctttgagtttttggaagtcggcttgtacctttctaagctgtgtcaaagcaggactctgaggaaggtacaaccataaactcagacgaattgtggcaagtgacgagcacatcatgcagacctcttgattttgtagccagcgttctacttcttctaacgtattaacgacttccacttcgtcgtcccaagccataatcagtttctgaacgattaagctgtcatcactccctggaagcgcatccatgtgcaatttcttgatagaatctctgttaaatctttttcgacatagtgggcatgtcgctgtagctgtcctttcaagacattctttgcaaaaaatatgcccgcatgctagggagcaagtcacttgtataataacacgaagtcttgtcaatcaatgaaaacttacgaatagcatatggctccttcagtgtggtctcctcttgccaggagtatgggtccaagcacacatcgcagcagctcgacgggtggacgataagcataagagcattccggagagtagttgagacaaaaagagggagctgcttgcgccactcaaatcatgtacacgaggcaggtaagtactattcgacaagaaaggaaattttgccaggctgtgcagcctgtgctcacagccacagcaaaagacttcattacataattgattataattttcaaatttgcggaaattctactctgctagtcgaccaatgcaatattcccctttcggtcaacattggcagcataatcttcgttttgggaggaataatgccagtcggttcacgtctacaatcaacgaactggactcccacccagcgtaaggtgggtattgccgctacagcggcatcgagactcccccatggcacctttagtgggtctaatacaagagaagtactggaaaagctaaagcaaaggataagatgagtaattccagctgcatttgtattctcaaaaaatctagataatcctaaaaccaaatcccaattagcatcgaagactcgtgagaggcccatagatagcccgagtcgctttaaggaaggcaagcggtcgagcgacatggtatctagaaatacgagatgtgaaacgactcgaacgtgaacaattcaagcgacaaaccaactggaggaccattagtagagatcatatcgaaccaaagttcttccaggtttgactgaacagactggtggtataataacgccccccaaggaagttcaggcaattttgattgtagcgaagatcggaagactgccagtttctgaagccgttcaagactgagattcggtagaatagcagtgatccacccaaagcacatcttctcgtcctcgatccactcaagcttacatgccgtgacgaacaatctagacggcatctctccgaaaggtattatactcggacgagtcagttccaaggcgattttggaaaatcccgtgcctcgtaagttcagatatgtcaaagagggagagataatgattcttgcattcaatagatcggtcaggtcggcgaaatgaaggaatagggaagtgactgaaggacgtctgatcgtggctctgattgagtcctgcagaggttggggtaattctctccaataagtcgggcgtgaggagttggaagcggctttccatatgaagcaggttagatcggtgagagaattcaagatatcaacgagggcggggtcgagctgtgtccagctaaaaccggagtcgagaatagataaggatcggatactcgaaacgagatgaggtgacgatcgaagtagcttcagtgttgcgtcacttgagcgttggtatcgtctatttccttctgccttcgaacaggatatgatgatattcgagaggagatatcgttggcagattggggtaaaggaagagcggacgagggacaaggatttcactgtagccttgtcctttctggctaggagttcgcgaattatagcttcagcgatgtctagcggcaggacaagcattgtgtgcgattgggcaagattctagctctggggaaatgagtatccttggtccggcgtatgccggatccgcttctgcaagtgagcgtgctttgagtctggtcactggtgttctggtagcggagtactaaattcaaaaataaagaatttggagcatgtgtcgaagttatgtttttactcgggctgtactaactttttgtggaatttgtctgtttctatcaattttttgtcaggcagaatggaaacatcggaaaagttgatggaaccatcgtgttagtgagttgacttccctccctcaaaacatatcacaataagtatgtacttagcagcctcgcgattccttggatagggcactaatctgttaaaggagagtcagtatctttcacatgttttccgcaacccaggagagtcccactatgtcagttccgcagtgggatccatacccaccagtcagtagagacgaaacctcagcaatcacgtatcagagcaagttgtgcggttctgttactgtacgcgatccgtacagtgcacttgaggtgccttttgatgatagtgaagagaccaaagtttgtggaaatcatgattccgtgtacaggatcctcaatgatcaggatataggcctttgttcatgcacaacgcaagtttgcgcggacgtatctagacgagattcctgacaggtatatgtctaatgtgctctttatcagtcactaacgcttttatttgacagagaaacgtggctgcaaacgctgaaagagagttggaactacagacgattcactgtccccaaacgcgaaagcgatggttacacctacttcgaatacaacgacggtcttcaatctcaaatgtctctacgtcgcgtgaaagtgagcgaagaggacaccattctcaccgagtctggacctggtggggaactcttctttgatcctaacctgctctctctggatggaaacgcggccctaactggctcaatgatgagtccatgtggcaaatactgggcttacggcgtttctgaacatgttcgtgtgttgtcataagggagcactttcaagttttactgatacaacatgagaaagggttctgattggatgacaacatatgttcggaaaacatcgtcaccgcatatgccttctcaagagaagggcaaagatcctggaagaatggacgacgttatccgttactctcgcttctttattgtatattggtcgagtgacagcaaaggtttggtcgcaatgtgcaaatggttatcaaagaggcctacttattcttaacggtctaggttttttttattctagatatccaccagaagatgacgagggcaaaggcaatacacctgcgcagaattgcatggtgagtttctttccacgctctatgtttactactctcatttccaacgccatcaggtgtattatcaccgcctcggggagaaacaggaaaaagatactcttgtctagtaagttcatgaggcatcaacacatggccttgcttatgaatattctgaaagtgaggatcccgagcatccattctggctctgggcactgcagctctcccctaggttagtgtttcccagcactcctgatcactgtattcacttagcaaatatgtagtggccgatatgcactcttgactgcaagcgtacgcaaaactcatactatatacacatctcactctaatgtttagctacacatagcgggatgctagtcatactcaactagccaaaattgctgatattgggacgagcgacattcaaaacggtattcaatggttgactatccatgatcaatggcaggctagatttgttatgtacgtctaccttctcttctgcttctcgacctcgactaacattctagttaatactgatctcattagaattggagacgatgattcaacaatttatttcatgacgaacctagaagctaaaaattacctagtagcaaccttggacattcggcattcagaagcgggggtcaagacactggtggctgaaaatcctgacgcccttctcatatcggcaagcatcctctcgaccgataaactcgtcctcgtctaccttcataatgccaggcatgaaatccatgtacacgatcttaacaccggaaaaccgatccgccagatattcgacaatttgatcgggcagttttcgctgtcgggacggcgtgacgataacgatatgtttgtctttcacagtggtttcacttctcccggcaccatatatcggtgagctcgttgataagacattgtggcaccctcgccgacattgtacaggttccgattgaatgaagatagtaacaagggcaccctatttcgtgctgtacaggttcctggcctcaacctgagtgatttcacgacagaatcggtatgcatacgtgcttctcttcatcgctcctattgctaagcaggatatgcaggtgttttatccatcaaaggatgggactccgtaatcattcttgctgaatctcttaaacatccgaagcctgataataaatttagtattcacatgttcattactcgattgaaggatacccccgtcgacggaactgcccctgtatatatttatggctacggcggcttcgccctggcaatgctcccgaccttctctgtctcgacgcttctgttctgcaagatctaccgggcaatgtatgtggtacccaacatacggtaattaattcatcatcgtttccctttcggtctctgaattggcttatgcttcagtggtggttcagagtttggagaatcatggcaccgggaggtgagtagtgtttaatttttgcgtaaaaaataactttgtctcaggcgcgttctacagggaatgttggacaagaaacagaacgtgttcgacgacttcaatgcagctacgaaatggctcgttgcgaacaaatatgccaataaatataacgttgccattcgcggaggatccaacggaggtgagctgagctgtaactgttcgaacctttgatcacagacattaagtgatgtggtttttctacaggagttttgacaactgcgtgtgcgaatcaagcgcctgagctctaccgctgtgtcatcaccattggaggtatcatcgacatgctcagagtttgtcaccatatccttcgttctgattcctcctaattcctcactcatccatgacattgcagtttcccaagtttactttcggcgctttatggcgttcggaatacggtgatgtactaaaatgtcaattggtctgaaaaaatctgaccctttatagcccgaggatccggaggattttgacttcatttacaagtgagctgaacagtcccgatacaagattgcttccttacccaaatcttgacaggtattccccttatcataacattccatcaggagatgtagtcttgccagctatgcttttctttactgctgcgtatgatgaccgagtttctcctctgcactgtgagtgggccaaaatcttgaggtcttatagaactctgatcggtttaaagcattcaaacatgttgctgccttacagtataacttccccaacggtccaaacccggttttgatgcgtattgatctaaacactggacatttcgccggcaaggtttgaaattttttactgtggtctcgcactttgctaattccatcccagagtacacagaaaatgctcgaagaaaccgccgacgagtacaggtgcgacttactctgttgtaacctacaattgtgattgatctcaatttagttttatcgggaaatccatgggactagttatgtgcgctcagaacgaacacgcgtcaaagcaatggtcctgtgtcgtgacctgaatcaaattgaaactatttaacgcctccctcgcccgaagtcccaaatatggatccgtaaccgtttaggcgccgcacctcacaaatagtttttcgtaacgatttagaaaagtcctacggtacaggatgggctagtcagttcttcactgaggctgtagaacgtataatagggataggtcagatatatgaaatcagtgtcgacttttgttacactactcaactacatacaacctttggaggtaaaacaataacaattagcaactaggtgctgatcgtctacggaacaataatccagcagtctggtttggagcgggtgaaaaagtataccacttatccgtgggtcgtctaccgttatgttccagcttgatgtcgtatgtcatcaagacgtgagcgagcatggtcttcaacagcgctgatactagaaagcggccaggactgaatacgttcacaaaatcatcaaaatatgcggatagttctttcacatctctcaccaagcatgccttccattaccaaacatcatgaaagtaggacccacggataccatttggtatcgaacagggtcatcttgttcttccctcagtcgagcaaatctgaacccgtcaaaatcagctggattggaatagtgcctctaggtatactctttatattttattccgagcagacgaaaatgaaaggcttacagcatcgtggtgtatcgcatagccaggggcaataatagagctccctgcgaagacagtcgtcccattggagaacgtgaaatccttcagaaccttgcggggcatgctcgctgatggatcgaatcatgaatcacacctgggagttcgcaaagtcaacgtaccttttccacctgcccgtaggcgtagagactccttgataaagctatcgagcttatacatcttgccaatggaatccttggatataccttcctctttgacaatcgattctacttcagctcgtagctccggaatatatactggaaatgtagccaggtcaaatagcgcttgtgtcaaggcctgaggttactcatgatataattcgaagtttatttttaacaacatggttcctcaccatagatgctgggcgatacgccgcaaacatggtcgctaacagtctgattgtgattgccttaatcgtagtcgcagataatttatcgctttccggcgttttctcaatcagccaagtgagaagatcattctggttacactctggtaagcagataccatacgatatttgagaactttgctcactgattttccagcccaatcggcgccgaacaatttttctttctcaaggcgttcttcgacaagaggagaaatgtgtttcattgcacgccgcagagttttgggaatgtagacatcaagaagtctggagacaattctacaacatcatttcaaaagagtatctagcaaaagcaggatttagaagactcacggcttgaggaaaccaggtgtgaaacctatgatcagagcagttttaacaagagcatctgagtatgcatcgttgatttcattgtagtctgaattgttgccttgatgcatatgcggtaaaccacgagcgcaatgatcatagcgagataaacaaacgtacaaagtgaaggaccaacgaataatctgttgacgactctgaggaccacaagttttccagtaagccaagccggaaaagtggtccagtctcttgacttgttagatgaaaaatctcgaaggacaagaatgaatttctcactatcagttggaggcacaagctctgagaaagctgcaatgacctcttccttgagctcaggaaaacgacctccaagatcgcgtgttagggagagatggataaatcgaaagtgataagggttctcgagaatgtgcggctccatcgtgtagtcagtttgcataccctgaaaaattgcttgagatcatgaacaatagaagtattgaggaacaacgaaagacctctttgatagagccatataaagacaattgatcgtcgctcgctttccggatgtcctcgatgagatcatgaccggagacaacaattgtcatctggaacaaagtaggaattgcgaacaactctccatagtgctaatggtagtaatcgttagctagtgaaacaattagcaagtagtagcattcatcggcacacctgatcgtagccttcctgaataaagtcgcgggttccgagtagaagctgaaaaaacccaatcacagttccaatccctgttggcccgacaaatattatgttcttgtacttcaaagagatgttatatgtctgggcatcaatgcatgggctcaaagcgtaaccatacctttgacttcctcaacttgccatcagcgatgcagcgagtaacggtgaggaatactaacgttataccaaatatgaaccaggtagtcgaggatggcataatgaggggaaagttgctcagtcgaaggattggtgacaaatttgaatctccgcgtcaaataatatcattcataaaacaatcggcggtgacatagacactcggtcaataactgtgatgaggataagaatcataggatcaagttggtgaaaccagggtgaaacgaagccgtggtggatatttacttgattgacatctcgcttggaagatagctgtagcctgtgggtgaaatcaagtctaatcaagtagataatcactgacaatgaaagcgatttctcagcgatacttcggtcataagaggtcatttatatttgaccatccataatatccactatggttcgcgccgccgagttatgtagacagaaagaatgcacgaaaagaatttcacttttagtagttctttttttttctggacgaaagagcttcggacctcaggctagcctcaaccagcctcaacctcagcgagttctccggcccgcctctcagcaattgatgcaatcaaaagtttccatgtataggaatttccttccccaatatggataatcttttcccgattgccatccgcccactaatccttttcatattctaacgatattcttgatacatactcttctagaatcgcgctacctggcggatttccagaaagaatatcgaaaagatcagaaatctccagatatccgatttgaatcctgtcaaatttttatatccttcttgccacctggactagtctggttcttggaaaatgacaacgttggagcaagagagctaagttaatcgttagtgaccacaagactcagctcccatgggccctgcagcatatttagcagaatcaaatgtcaagctgccctgaattttaaagatctatgtacgcagttagattttcccttgtggctcgctaaatacatactgctaactgcatgcgaatgatactctgcagccaatcacatgagatgcagaacatctatttgaacggggcttatgcctttggctttgcttctcaccaccaccaccatacgactgagcccacaccatgactgataccacgaagattaaaaccgttcgaaagttccacgacctcctgaacgaaggaaagcttgatgaggctcatgagctcttggccgaagacttgtctgcgcacaccactgcgcatatcgccgcaggggcacccccgatgagtaaagcagaggttcttgaatctcatcaaggcattcatcaagagctggaagctaaggttagtctctcagtctcctctacactgttgtaggggcttcaactgagtatcatctagacaaccatcaccgacgccaaggaggtcgatggaaatgttatcgtagtggtgagtcataatgatcatataaaacatcatgtaaaaattctaggtttaacgcgttctaaatgattagggaaagtccactttcaatcacgacactatcaagtatcattaccttctcccattcttattgcgactatctttgatgcctttttagggaggttccgttcaaggcaacctatgaattcatcgggaatcagattggcaaagtccatttcgagacggatcctaaccacaaagttaaatccttcagcctctaggagctctgcgggatctttgaattgaatctgtgggaccgtggatgaatttcccggtggtcaggaatacatatgtagtctatccgttgttttgaagagcacgtatttgttgtttgtatgatatggatgcaagaaaagttgaagttgtgtattcaaataacattgactcgtgaaaaaaaggaggggactgcgaaagagcagtgctattgagtacggtacacgagtgacaagtagtacgctgtacatgtagatatcgcgtgaaaagcaacctcgcaattgtatatgagtgaaagtgagtgctgtcagagcagatcacttggctacaagcaactttcaagcaagtccgatgaaagaattgtacaatacgaccaatcggatttgatgctaagtcctattctgtcctagaggctattgttactacttccatacctcctgccaacaacggacaatctgggctcctctgttgtactttcccaacttgagctactccatattcttctgcgttggagtaattcccactggcagggacccaggcactatcatcatagctataggaaaggctcaggagaattccgatatttgacagcaaggatgttggcattaacgctcgctacactcatcctagccccgttttgcaattctcttctcaaccataatcttatggaatattctacagatagatcatcagtgtattcattgcgtccgaaaatcgcatttccaacagacggtgaaatcttcctgcaggatgagcgatacttcatttattgaagtgcagagatatttcagatatataaagcctttgaaaagtagccaactgtgattcagcatcacctacatagtcatcacaatggagactcctaccttaaacaaatccggctccctcactatcgttggcactggcatcgagagtattggtcaaatgacccttcagaccttgtcttacatcgaagccgccgacaaagttttttactgtgtcatagaccctgccaccgaagcattcatcctcaccaagaacaaggactgtgtcgacctgtaccagtactacgataatggcaagtccaggatggatacctacacccaaatgtctgaggtgagatgtttcttgtcaaatgttttctttgtgttctgtatagagctgacatcttcaaaggttatgctcagggaggtccgcaagggccttgatgttgtaggcgtcttctacggacacccaggtgtgttcgttaacccttcgcttcgagcgctcgctatcgccaaaagtgaggggttcaaggctaggatgctgccgggcgtatctgcggaggactgcctttatgcagacttgtgcatcgatccttcgaaccccggatgtctgacctacgaagcatctgacttcctcattcgagagaggccaacgaacatttacagccattttatccttttccaagttggatgtgtcggtattgctgacttcaacttcactggatttgaagtaagtatcatcaatctccatgaggtatacatttaataaactctttgttctagaactcgaaattcggaattcttgttgaccgcctcgagaaggagtatggcgcagaacatcctgtcgtgcactacatcgccgctatgctccctcacgaggatcccgttaccgatcagtggaccattggacaactccgtgagccggagttctacaaacgcgtcggcggtgtttccactttctacatcccgccaaaggagcgaaaggagattaacgttgatatcattcgtgaactcaagtttctccctgaggggaaagttccggatacgcgtactcagatttacccacccaaccaatgggaacccgaggtaccgactgttcctgcgtatggatcaaatgagcacgccgctatcgctcagttggacactcacactcctcccgagcaatatcaacctctcgctacttccaaagccatgactgatgtgatgaccaagttggcgttggatcccaaggcgctcgcagaatacaaggctgaccaccgagccttcgctcagtccgttccggatttgacggcgaatgagaggaccgctttggagattggagactcctgggcgttccgctgcgctatgaaggaaatgcctatttcactcctagacaacgccaagcagtcaatggaagaagcctccgaacaaggcttcccgtggatcatcgtcgttggtgtcgttggtgtcgttggttctgttgttagtagcgcctgattattctgattgcctctcaaattcaaaacactgtaacaactacgataagtattctcatatgtatgtaaagaaatattgtcagtaaatatgtcactcacgaggaatgcctgagcctcgaaatatcacatatctaacattgcgtgtattattgtttccatacaatcaacttccttctacacggacctaatgaacaattagtagtcagatgcatctcgcctcctctcgcgcggttgctcaagctggcccctgcttgcccctcctactatgtagcatgataaggcatcaagatccgatgtgaacgtaaaaacctatcagaatcaggaatgaatataaatccctgatattcaaaggttcgacgaggaacctcagggaacttcaaagcctgaggtttggtctgggttcggttacgtaaaacgacgtcacgaatctcgaaattttcaaagcttcatgttgatgatctgtgaggagagcattcctgtgatttcgacacgacagcaaggatttgaaggtgtacatgggctgaacagacgctttatattatcacagcaaagtacatagcaatcatcaaagactcaccttaatctacactgatccaatcaccgcctagttagttgacaggtaacacgattcgctaggatgacaatgatatgcaactcaatcatcacaagacatgcctttgccttgcattttccttttcttgagcggaaatgaaagtttggaaagagatatataaactgagctaaactgtcacgtacgtgggaatttcaaataatatggcgatattttcttccgttttcctgttcctcctagcggtgggacttgctctgctaacaattcttacgaggcgcaaaatcaaggaaagctatatcggcttccgtgctatagcttacagctatgacttgatgtcatctagcttcagacccggccaaaacacgtatcatttcactattcttggagtatgtactagcagtattattccatcttaccgctgtgatgtttaccactgaagtagaatcctgtggttggattgagaggtcaagaagcgaggaaagtgttcttcaacgaaaggaacttcagtactagtgaaggatacaaggtgttcagcaaagcagctcctgtatgcgaactgcaaatctccttatatgccattgttgaagctttttaacaggttccagaactagctaaaacgttactttctgcagaggaaggccatggacttcacttcaagaagcaacttgaaattctcgtgaacaagaatcggttagctgacagtaagcagcaaatagctacgtgtagtaggctcatatttaaatacacggcagttatcgcaactatgctgggggatatgcagcggctcacggctacctggaataataaagggtcgatggatccttttgacaaaataaacgacgtacgtgttgccgattttactagacctcgattaaaacttcgtctacatagatcacctttcatgttaccgcgcatatgatcactagcaacgacttcgtttcagatccagtgaatttctctcgatttcaaacccatttcttcacccaactcaagaataacactcccgcaatgattttcttaccgtggttctatcgtcaatcacaacgagaaaaagaaatagctataacttccctcttcacgaccttaacgagctatatcgaagctcgagaaaaggacaaaatttcgacttctgaacccatcgatctgttacttgaccacgggatgaattcacaggagatcatccagttcattctcggattcctgtttggaggtgtttacaacgcaactaaagcaagtacgcgtccaataccttttcaagttttatgtgattttaccgcgttgtatacatagtctcctggatacttatctatatctcgatgcattcccaatggaaaatggccatccgttccgaggtagaaaatgtcctctccaaacattctttcgatatctccaagccgctatacagcagactctctgaagtaccgctcgaggcctgggaggaatcgctgcctacgctcgacctcgtcatttccgagacatttcgcctggtcatgaatcaaaccgtcttacggcggaatataggcgaagatgtcgaaatacctggcggacagggccatgttccaagtggagcattcgtagcatatagtaatgccgatgctcatctggagcccgaaatatacgacgatccttggtcattcaagcctgatcgttttgaacatggaaaacagaaggtactctcaaacgacttcttgggatggggcgctggtaagcaatgttttcatatgttgatgccaatgaatttgcaaaccatccaataccagggagacaccgctgtgtaggattggggatcgcaaaactgacgatcaagacgattgtctgtttattcgtgatgacctacaattacgatataactcggacgaagcgcaagtctcacccgacaattccgattcctgatcataacaaggtaaatgaggtgagtgtcttgaagaagtgtacggcctacagattcattaaaatcttaatacgcaggcacgcccaaagggtgaagtgattactctcaattaccaacgtattgaacaatctgtttcaatataagttggatgttgtgactcgtactgtaccacacgaaacctgagcacagagaaaggacaggaagatgctctttatgtcaacacttttgagtgttacaaatgtgtcctcttgagcctgctgccggaataaaaccagggtactttggcgcggagaccatgtcttttgacttcggtgtatatttgaattgtaatatcactgccagacaacgagaaaatactgcagccaacaacctgaatccggggcatatcgaatgagatcaatttcgaaaatttcagagcactatagttccatatggtacatacaacaagatctctgaggtcgtggtttgagccatctatattttcacaacttcttcgagttcacgatctgtacaaattcgggtttcagggaagcacctcccacaaggaaaccatcgacgtcgggtagagtagctagaacatgggttgaggtcgatcagagacagatagatattcggcaggaaaaatggttcaaaacaaaaagctcacaaagttctttgcaattcccagcattgacgctacctccgtagatgatcctggtcgattcagcgacacctgacgagacagctttggatagatacgaccggatatctttgtgagcctcctgcgcctgctgtggagtagcaactttgccagttcctatggcccacacgggttcgtaggcaacgacgattttacttgtagataagatgggaagggagataagacgctcaacgtatgaaagatctacacaaaatctcacctccaatcgctctcctttaatacatctacgacagctttgagttgttcctcacagacttttgcggtctctccggcctcgcgttcgttgagagtctcgccgatacagagaataatgttgagtcccttatcgagagcagcgcgagtcttttgcgcaacgacttcggaagattcatggaagagggttcgacgctccgagtgtccttgaggttaacaagaaaaacatatgagtattcggctactgtcatcatacatagttaacgcaccaaggataacgtagggtatttcagaatctacaagttgtgcaggactgcatatggcatctgagttcgaagtttgcaatgtaaattcggaatcgcgaacctgatctctccggtgaaagcaccagagtctttgaaatagcaattctgagcagctactttgatgtccttccggatggacccttttacagggataagatatagagctggtggagatatgacgacctctatatatcaagaagaacacacgggcaaagaataaggtcagatgtttaatgagagtgtgagtgagtaagacgtgccagtgttaggatcaagctctgcttcgttcaaaagcttcaccagagctactttgtccttccggctagccggattcatcttgaaattgcctccaacaaagaattggcgcgccatgtagttgctcggataggataatgagtgaagtgcaagctgatcgattgctggtggctcaaagtttatttgttctcacgatttgcttcgatcagttctgttccgattacagtatcgtccttcttcgccgctatagtccttgcacgtattctcaatctggatgttatgcaatcgccactgagctgaatgtgcgtcgttagtaccgtatgcttcaaacgttgcggacttgatgctactcgatgtgctttaacaggctttaactcatctttcttcgtgcattaatatttcaggttaactttgggatggaccttgatagacccagtttcacggaacgatctcgaagtaatgggccaacggcattgacagaaaatggatacgacccgccggttaggttgacggttatcagttccgtttcagtcattcttcctgacacattcatattgaacgcgtgcgtgttattttgccttatttgaaccaaggtcatattataactacctagctgactattggttcctcgaagaagtggattgatctttcatgcacactgacgacacctaactcatcgtttcaactgcttaaagcattgcgcaacggtgatcgtctctccctcgataacatgaaactttacaatcctttatacccacctcccgctccacctgcatttggcagagatacagtcctaccagaaccaaaggcatcgtgtgtcattgtccatcttgatgtcgtctttgcctccctcacgcttggaaatttcacagactactctcccgcctcgtatttcagtggttgtcgccgtttctagctgtaggattctctcggcctctggagaaggacggtgagctatcgacacgaaatctgttcattgcttacctccttgtcattcgattcagatttatggcaactcccctctgcacgtctcaccgcgaatataaccgatagaatcgagacgaatttctttcaacgatgcccccctgaccagaggcctcgtcatttacgtcaaaagctccgtcaattttgggaagctgaagtgacggacgcgattccagagcaggacaatgacaacgatagcacgtacgaaggctcgtcagatacgcagaagaccgacgagaagaagagcttcaacaagaagaagaaaaacatagaacctaaatatgattcctcattattacaagccatatacataacttttaaagccgaatggtggatctctggaatgctcaagttaatttcaggttaccccgatttccttcttctctgctcgaggtcgcaaatgctcactcctaactctatctgctcctttagatacactcaagaccacgacccctctcgtcaataaagttattctcacttggcttacagactcgttcatattctacaaggctacgccatcagaaagatctgagcttggattgacttcgcctcgtggtatcggatatgggattggacttgcattcgctttgttcgttatgcaaggtatgtacttgtttatttctcagaaatttactcacatctgccttagaaagcgctagtttggtgagacattgacaacccgttataattttaccccatctaaatttgctctagatgacgaatcattacatgcagagtatgtcaatatcggtgcagttgagcaagaactttactaacccagcccctgccttgtaaacagtaagcatggccaacggattagccatcagaacgggcgtaggttgtttcacaacagtacacattccttcaatttgaacatttttcgtagatcatagggaacatattccgcaaatccttgcgtctttctgggcgtgcaagaaaccagcatagcgttggacaggtgcacattcatagaattcccgtattttacaccgagctcaaacagataacagataaccacaatgatctccactgatgctacgaggttggacacattctccgccaatgtccacaagtgtgttatacttgtccttttgtatgcttactcctgacatgtaatctatgaagtatttgggtatctccaatccagatagccattggtatcgggcttctcattgcaaacgttagtatccttacctgcagccgcatttatgacgaagatttgatattgtatctacttttatggcacagctgggatattctgcacttgtgggattgggtgtaagtcatagtatgcttatcccgtgttgcatgcaatcgcgtctaattttctggatctaggtacttctcttgggactccctttgcaattcctttgcctgaggataatgttcatgaacagaagaaagggagtgaaggtcacagatagtagagtccggcttaccactgaggtatggataataatttcatgcacttggatacctatgaaccaagttttgcttccacaggttctgcaaggtatccgcctcatcaaagcatatgcttgggagggattctatgtttcccaaatctacaacttcaggaagcaggaagtggcacggattcggactgcttcgtaagtaaattatctctgcatgagtttgtcactgttcttgtgttaacttcaccatccaggatagcccgcgcagttctcattgccctggtcacgtttatcccagttcttgcttctgtcctctcctttgtgagcttccgttgtgtatagaaccagaactacgtattgatcaacatttttagatcacttatgcattaagcggccatgatctcaatgttgcgattattttcagctctttacaattcttcaacgtcagttaacatccgtctgtagtgtagctgtgacactgaccctgacctccgacctgttttcgtagattattcgtgctcctctcatctttttccctatcgtcctctcttcattgtccgacttcctcgtcgccatgggtcggattggcaaattccttaaagcagaagagcttgcagaatcttacaccatcgacttcgaacgtaaacatgctgtggaagtcgatggcgacttcagttgggaatctgcaggtaaaatagtagaaacaaagtttgaaaagggtgcaaagaacgaccatgtcccaaactccggtgtgaaatccaaggaagatggaaagcctgcaaaggcggtctcaaaaagcacccaagacgaaactctacccaccacttttcaagatatcaaaacgacgcaagacattgatagtgatcccctgaaggaagatgaacccttcgtattaaggaacctcaagttcaacgtcccgaaaggttcttttgtcgccatagtgggcactgtgggttcagggaaggtgagacccaacccttataatcccatcccgccacgtctaatcgtgccctttctgtgcagagttcaattctgaacgctttgataggggaaatgcggcgaactcgaggagaagtagttttcggtggaactgtgtcctatgtcccacagactccatggataaggaatgccactctgaaagagaacgtgctcttcggcatggaagaagatgataaaaagtaagcattgttcattcttcctgatggcactgacatgacatgacaactccgtaggttcaaagagatcatgcgcgcttgtaatttggagcacgatttagaactactaccgcacggcgagtatactgaaattggcgaaaagggtatcaatttgagtggtgcgagcattaaccgaacgactaaaaactactgctcaattcatgattctaggcgggcagaaggtagcaaccatcccatttcgtatattcgaggttgaatttgtctcactctctcgtttcataggcccgagtttcgttagcgagggcggcatactcagattcagatatcgttcttctggatgacccattatccgctgttgactcatatgtgggcaaatctatattgacggattgcttcctggacggtccactggcagacaaaacgcgaattctggtcacacatgctttgcacgtgcttgacaaaacagattacgtttacgtggttgaccagggagaaatcaaagagcaggggacattccaggtgtgtaaaattcatactagtgtgttgcagctgtcgactcatgtctccagacgcttatgtctgccaatggccttttcacgcggatcatggatgaatatggaagcttaaatcacgagaacgaaagtaacagcgagaagccaaggacggaggatgaagtcgtaaatcctatacccgacaacaaggctgcaaatgctgctcttatgcagacagaagaacgtaacattggcgctatcgggtatcaaacgtatgcgaaatacctaaaattcgccggagggatttggtgggcgcctttcattcttctcctgttaactttaacacaaggagcgcaaggtgcgtcagttcaacttttgatcgagagttcagctgaaattccttcgaactttagtcgcaaataatctgttcttgggcttctggactgcggaaagcattccaggttttggccaagctcagtatatgggtgtttatgcagcgcttggtacgggaggccttgagcattgatatatatgggttaaccgcactctctaggttttgctcaagccctgttcacgtttctgttgtccttttcgtttgcgtgagtttatcggccaattatttgctggatcgagatcgatgaccagatgtacgcttgtagtattgctggccttgtagcaagcctcaatctcttccaaacagcactccgtcatgttttacgatctccaatttcattctttgacactacccccatgggtgagtaagcgttcattcgcagtgtccttcaaacttagaaatttttcattaggtcgaattctctcccgtttatcgaaagatcaagatactttagactccacccttacgatgaccttgatgcaatttctgaacacttttagttctgtcttgggcaccgttgcactcgttttctacacttttcctcttttagggatcatctttgcacccatgacagtgttctactatttcatctccatttattaccgacggtcatctgtcgaaacgaaacgtttggattcgttgatgcgttcggcactatatagttcttactcaggtaatctgcactcgtctctgattttctgacatcagttgacagggatactccattgtagaaactctcactggtctgtccactattcgtgcatatcgcgaacaggcaagtgtctccccctaacgctatgtccctcaatctaaacagatgccacaggcgcgatcggttttaaatgcgcaaaatggcctcgatttggagaatcgggcgtattatatgactatatccatccagcgatggctatctataagattagattttctgggaaatattttgattttgggaattgccctgtttgccgctggtttcagagaaacagtgaatccttcgaagattggggtcgttctttcatacacgcttagcagtaagtcattaatttcactctatcagacaagctcctgcctgaacttgtttttgatgcagtaactcagatattctgtatgttgcatcttgtcgtgttctatctcctttcgatccattgagcctgctttcatagcggaaatggtcagccaatacgctctgagcgagcaggtaatattcaccccagttccagctttgaaccaccctaacacccaagtagaatatgaatgccgtggaacgtatcttggtatatgcagaattaccaagcgagggtgacccagtgacgccgcatgatccgcctccatcatggcctcatgcgggacgcgtcgaattcagcaatgttcagctggcctatcgaaaaggtcttcccctcgtgttgaatggcgtttctttcaatgtgcagcccggagagaaggtgtgttggcgaaatacttccccacgatctcaatattaattctgcgtatagattggcatagtcggcagaactggggctggtacgatgacagtgttttggaaataccgacatcactcacatggacaataggtaaaagttctcttatacaggcgctgctccggtacgatgccttatccgatctcaatccagtgcgccactaacatcatgcattagcatggttgaacttgagggagggaaaattgagatcgatggttttaatacccgagaaattggcttggacgttctaagagggaagttagctctagtgcctcaagatagtgttctttttcttggaacactgcgcgagaatttgtaagggtctattacatgacatcctatgttcctcacggttttaatatttcgatagggacccaatgggcactcgcacggatgcggagctgatctccgcattgcaacgagcttggttgttgccgcgcgatggaacgaaggatcccgtggctgaagcaaaattcagtttggacgccaccgttggcgacgaaggtgagaatccgctggaattggttctgcattgaggacctctaaatacgtccggaaggctccaattttagtgcaggagaaaaacaactcttagctctatgtagagctcttgtcaagaaaagtcgaattatcatcctcgtcagtaaaagttttcagcacatacatctccatactaacactctcgatcatgtaaaggatgaagccacaagcagtgtggatgtggaaacagatggaaaattacagagaactattcagacagagttttcgacctcaagtcttctctgtatagctcatcgcttgaataccatcggtaaacccctacttccaatttcctaccgcgtatatacctatctgacggattcatgtagcatactatgatcgtataatcgtgatggatgctggacaagtcgccgaattcgatacggttctaaacttgttcgaccgtgaagactctattttccgatccctctgtaatgaagccaacctacaacgttctgatattctacgcatacgagctagttatggagatgtagccccgatagagatgcccgcataactgaaaggccaagtaaatcaataaatagagatattttaaacttgtagaccttaaaaacgtagacaaacggatattacttcttgaggccccactattttcagcttctacatctatatcatggaatctcttaattcccgctacctttccagaggtgaaatagccttatagggccaacttttcgtttctttgaccttttttaatcaatatttaattgtacaatttttacaagtctctgtagtcgacaatatatctatatactgtcttcctattctcgcaggagaattttacttacaaatggtatcatgggccctggtagcaggccctgccgctgtctgaaaatttatgtccagcattttgcagggcaaagaaatctattacccgaatatacgcaaggttcc

(19) United States
(12) Patent Application Publication
(10) Pub. No.: US 2020/0199640 A1
(43) Pub. Date: Jun. 25, 2020

(54) PROCESS FOR OBTAINING OMPHALOTIN IN A HOST CELL COMPRISING POLYNUCLEOTIDES ENCODING FOR POLYPEPTIDES INVOLVED IN THE OMPHALOTIN BIOSYNTHESIS

(71) Applicant: TECHNISCHE UNIVERSITÄT BERLIN, Berlin (DE)

(72) Inventors: Sascha RAMM, Berlin (DE); Agnes MUEHLENWEG, Berlin (DE); Annette POCH-HASNEK, Berlin (DE); Eva MOESKER, Neuenhagn (DE); Roderich SUESSMUTH, Berlin (DE); Bartlomiej KRAWCZYK, Biberach an der Riss (DE)

(73) Assignee: TECHNISCHE UNIVERSITÄT BERLIN, Berlin (DE)

(21) Appl. No.: 16/608,825

(22) PCT Filed: Apr. 27, 2018
(86) PCT No.: PCT/EP2018/060835

§ 371 Date: Oct. 26, 2019

(30) Foreign Application Priority Data
Apr. 27, 2017 (EP) 17168405.3

(51) Int. Cl. C12P 21/02; C12N 9/02; C12N 9/58; C12N 9/10; C12N 15/81

(52) U.S. Cl. CPC C12P 21/02; C12N 9/0071; C12N 15/81; C12N 9/1007; C12N 9/1025; C12N 9/58

(57) ABSTRACT

The proposed solution refers to a host cell for the heterologous synthesis of at least one omphalotin compound comprising polynucleotides encoding for polypeptides involved in the omphalotin biosynthesis, an expression vector comprising said polynucleotides and a process for obtaining at least one omphalotin compound from said host cell.

### CROSS-REFERENCE TO A RELATED APPLICATION

This application is a National Phase Patent Application of International Patent Application Number PCT/EP2018/060835, filed on Apr. 27, 2018, which claims priority of European Patent Application Number 17168405.3, filed on Apr. 27, 2017.

### BACKGROUND

The disclosure relates to a host cell for the heterologous synthesis of at least one omphalotin compound comprising polynucleotides encoding for polypeptides involved in the omphalotin biosynthesis, an expression vector comprising said polynucleotides and a process for obtaining at least one omphalotin compound from said host cell.

Omphalotin A (Scheme 1) is a head-to-tail cyclized all-L-dodecapeptide produced by the basidiomycete Omphalotus olearius. It displays strong and selective activity against the plant pathogen nematode Meloidogyne incognita and shows no further phytotoxic, antibacterial or antifungal activities (A. Mayer et al, Pestic. Sci. 1999, 55, 27-30).

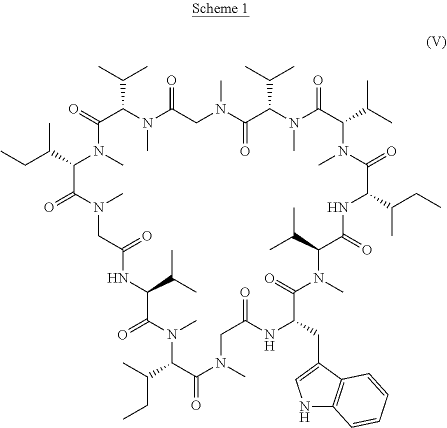 [SMILES: CCC(C)[C@@H]1NC(=O)[C@H](C(C)C)N(C)C(=O)[C@H](Cc2c[nH]c3ccccc23)NC(=O)CN(C)C(=O)[C@H](C(C)CC)N(C)C(=O)[C@H](C(C)C)NC(=O)CN(C)C(=O)[C@H](C(C)CC)N(C)C(=O)[C@H](C(C)C)N(C)C(=O)CN(C)C(=O)[C@H](C(C)C)N(C)C(=O)[C@H](C(C)C)N(C)C1=O]

The challenging total synthesis has been accomplished by Jung and co-workers facilitating a profiling of its bioactivity (Angew. Chem. Int. Ed Engl. 2002, 41, 2307-2309).

In addition to the main compound omphalotin A, the derivatives omphalotin B to I, display various modifications e.g. acetylations, hydroxylations and an unusual tryptophan modification. A unifying feature of all omphalotins are nine N-methylations of the amide backbone. First isolated and described in 1997 the biosynthesis of the omphalotins was suspected as produced by non-ribosomal peptide synthetases (NRPS), as it has been described for cyclosporine, a structurally related cyc/o-undecapeptide with seven N-methylations and one D-amino acid (D-Ala).

Due to its high potent activity against nematodes, it would be desireable to provide an approach to obtain omphalotin and derivatives thereof in an easy manner.

### SUMMARY

The object underlying the proposed solution is therefore to provide a reliable biosynthetic approach for synthesizing omphalotin and derivatives thereof.

Accordingly, a host cell for the heterologous synthesis of at least one omphalotin compound is provided comprising polynucleotides encoding for polypeptides involved in the biosynthesis of omphalotin and expression-controlling elements operably linked with said polynucleotides to drive expression thereof in the host cell.

Specifically, a host cell is provided comprising a first polynucleotide encoding for a polypeptide with N-methyltransferase activity and omphalotin precursor peptide activity, consisting essentially of Seq ID No. 1 or a polynucleotide with at least 70%, preferably at least 90%, more preferably at least 95% identity thereto is provided. The polynucleotide of Seq ID No. 1 comprises introns. The corresponding polynucleotide (cDNA) without introns is represented by Seq ID No. 2.

Seq. ID No 1 and Seq. ID No 2 comprise preferably polynucleotide ophMA of Omphalotus olearius encoding a polypeptide with N-methyltransferase activity and omphalotin precursor peptide activity. Thus, polynucleotide ophMA encodes for a fusion protein between a methlytransferase OphM and a precursor peptide OphA.

The 3′-region of the polynucleotides of Seq ID No. 1 and 2 comprises in each case a polynucleotide sequence encoding for a leader sequence (represented by Seq ID No. 3), a polynucleotide sequence encoding for a core sequence (represented by Seq ID No. 4) and a polynucleotide sequence encoding for a follower sequence (represented by Seq ID No.5). It is to be understood that any polynucleotide with at least 70%, preferably at least 90%, more preferably at least 95% identity to Seq. ID No. 3-5 is also covered by the proposed solution.

The core sequence encodes for a polypeptide forming the backbone of the omphalotin compound. Specifically, the core sequence encodes for 12 amino acids that—after methylation—form the backbone of omphalotin A; either in cyclic form or also possibly in linear form.

The host cell comprises also a second polynucleotide encoding for a polypeptide with prolyloligopeptidase activity, consisting essentially of Seq ID No. 6 or a polynucleotide with at least 70%, preferably at least 90%, more preferably at least 95% identity thereto is provided. In particular, Seq ID No. 6 comprises preferably polynucleotide ophP of Omphalotus olearius encoding a polypetide with prolyloligopeptidase activity.

The polynucleotide of Seq ID No. 6 comprises introns. The corresponding polynucleotide (cDNA) without introns is represented by Seq ID No. 7.

In one embodiment the host cell also comprises a polynucleotide encoding for a polypeptidewith monooxygenase activity, consisting essentially of Seq ID No. 8 or a polynucleotide with at least 70%, preferably at least 90%, more preferably at least 95% identity thereto is provided. In particular, Seq ID No. 3 comprises preferably polynucleotide ophM1 of Omphalotus olearius encoding for a first monooxygenase activity. The polynucleotide of Seq ID No. 8 comprises introns.

In another embodiment the host cell comprises another polynucleotide encoding for a with monooxygenase activity, consisting essentially of Seq ID No. 9 or a polynucleotide with at least 70%, preferably at least 90%, more preferably at least 95% identity thereto is provided. In particular, Seq ID No. 9 comprises preferably polynucleotide ophM2 of Omphalotus olearius encoding for a second monooxygenase activity. It is to be understood that Seq ID No 9 (or ophM2) comprises introns and Seq ID No. 10 (ophM2a) represents the cDNA of ophM2.

in a yet further embodiment the host cell comprises a polynucleotide encoding for a polypeptide with acetyltransferase activity, consisting essentially of Seq ID No. 11 or a polynucleotide with at least 70%, preferably at least 90%, more preferably at least 95% identity thereto is provided. In particular, Seq ID No. 11 comprises preferably polynucleotide ophC of Omphalotus olearius encoding for an acetyltransferase activity. It is to be understood that Seq ID No 11(or ophC) comprises introns and Seq ID No. 12 (ophCa) represents the cDNA of ophC.

In still another embodiment the host cell may comprise a polynucleotide encoding for polypeptides with activity for omphalotin synthesis consisting essentially of Seq ID No. 13 or a polynucleotide with at least 70%, preferably at least 90%, more preferably at least 95% identity thereto is provided. The polynucleotide of Seq ID No. 13 comprises preferably at least one of the Seq ID No.1-12. In particular Seq ID No. 13 comprises ophMA, ophP, ophM1, ophM2 and ophC.

It is to be understood that polynucleotides from other sources may be included into the proposed solution as long as the polynucleotides have at least an identity of 70% to claimed polynucleotides.

In one embodiment, said polynucleotides may be found in Lentinula edodes and may comprise a methylase according to Seq ID No. 20, a prolyl oligopeptidase according to Seq ID No. 21 and a cluster with biosynthesis activity according to Seq ID No. 22.

The “polynucleotides” or “nucleic acids” of the proposed solution may be in the form of RNA or in the form of DNA; DNA should be understood to include cDNA, genomic DNA, recombinant DNA and synthetic DNA. The DNA may be double-stranded or single-stranded and, if single stranded, may be the coding strand or non-coding (antisense) strand. The RNA may be mRNA.

The solution also includes polynucleotides where the coding sequence for the polypeptide may be fused in the same reading frame to a polynucleotide sequence which aids in expression and secretion of a polypeptide from a host cell; for example, a leader sequence which functions as a secretory sequence for controlling transport of a polypeptide from the cell may be so fused. The polypeptide having such a leader sequence is termed a preprotein or a preproprotein and may have the leader sequence cleaved, by the host cell to form the mature form of the protein. These polynucleotides may have a 5′ extended region so that it encodes a proprotein, which is the mature protein plus additional amino acid residues at the N-terminus. The expression product having such a prosequence is termed a proprotein, which is an inactive form of the mature protein; however, once the prosequence is cleaved an active mature protein remains. The additional sequence may also be attached to the protein OphMA and be part of the mature protein. Thus, for example, the polynucleotides of the proposed solution may encode polypeptides, or proteins having a prosequence, or proteins having both a prosequence and a presequence (leader sequence). The polynucleotides of the proposed solution may also have the coding sequence fused in frame to a marker sequence which allows for purification of the synthesized polypeptides.

The solution is considered to further provide polynucleotides which hybridize to the hereinabove-described sequences wherein there is at least 70%, preferably at least 90%, and more preferably at least 95% identity or similarity between the sequences, and thus encode proteins having similar biological activity. Moreover, as known in the art, there is “similarity” between two polypeptides when the amino acid sequences contain the same or conserved amino acid substitutes for each individual residue in the sequence. Identity and similarity may be measured using sequence analysis software (e. g., ClustalW at PBIL (Pôle Bioinformatique Lyonnais) http://npsa-pbil.ibcp.fr). The proposed solution particularly provides such polynucleotides, which hybridize under stringent conditions to the hereinabove-described polynucleotides. As herein used, the term “stringent conditions” means conditions which permit hybridization between polynucleotides sequences and the polynucleotide sequences of SEQ ID No. 1-6 where there is at least about 70% identity.

Suitably stringent conditions can be defined by, e. g., the concentrations of salt or formamide in the prehybridization and hybridization solutions, or by the hybridization temperature, and are well known in the art. In particular, stringency can be increased by reducing the concentration of salt, by increasing the concentration of formamide, and/or by raising the hybridization temperature.

For example, hybridization under high stringency conditions may employ about 50% formamide at about 37° C. to 42° C., whereas hybridization under reduced stringency conditions might employ about 35% to 25% formamide at about 30° C. to 35° C. One particular set of conditions for hybridization under high stringency conditions employs 42° C., 50% formamide, 5×SSPE, 0.3% SDS, and 200 μg/ml sheared and denatured salmon sperm DNA. For hybridization under reduced stringency, similar conditions as described above may be used in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art. Preferably, hybridization should occur only if there is at least 95%, and more preferably at least 97%, identity between the sequences. The polynucleotides which hybridize to the hereinabove described polynucleotides in a preferred embodiment encode polypeptides which exhibit substantially the same biological function or activity as the mature protein encoded by one of the cDNAs of SEQ ID No. 1-13.

As mentioned, a suitable polynucleotide probe may have at least 14 bases, preferably 30 bases, and more preferably at least 50 bases, and will hybridize to a polynucleotide of the proposed solution, which has an identity thereto, as hereinabove described, and which may or may not retain activity. For example, such polynucleotides may be employed as a probe for hybridizing to the polynucleotides of SEQ ID No. 1 to 13, respectively, for example, for recovery of such a polynucleotide, or as a diagnostic probe, or as a PCR primer. Thus, the proposed solution includes polynucleotides having at least a 70% identity, preferably at least a 90% identity, and more preferably at least a 95% identity to a polynucleotide which encodes a polypeptide of SEQ ID No. 1-13, as well as fragments thereof, which fragments preferably have at least 30 bases and more preferably at least 50 bases, and to polypeptides encoded by such polynucleotides.

For example, polynucleotides according to Seq ID No. 14-19 are provided that are used as PCR primers.

Seq ID No. 14 and Seq ID No. 15 comprise oligonucleotides applicable as PCR primers for ophMA sequence. In particular, Seq ID No. 14 may be used as forward primer (ophMAfw) and Seq ID No. 15 may be used as reverse primer (ophMArev).

Seq. ID no. 16-19 comprise oligonucleotides applicable as PCR primers for ophP sequence. In particular, Seq ID No. 16 may be used as forward primer (ophPfw) of a first ophP fragment and Seq ID No. 17 may be used as reverse primer (Sallrev) of the first ophP fragment; Seq ID No. 18 may be used as forward primer (Sallfw) of a second ophP fragment and Seq ID No. 19 may be used as reverse primer (ophPrev) of the second ophP fragment.

The terms “homology” or “identity,” as used interchangeably herein, refer to sequence similarity between two polynucleotide sequences or between two polypeptide sequences, with identity being a more strict comparison. The phrases “percent identity or homology” and “identity or homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Identity or similarity can be determined by comparing a position in each sequence that can be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences.

The degree of sequence identity is determined by choosing one sequence as the query sequence and aligning it with the internet-based tool ClustalW with homologous sequences taken from GenBank using the blastp algorithm (NCBI).

As is well known in the art, the genetic code is redundant in that certain amino acids are coded for by more than one nucleotide triplet (codon), and the solution includes those polynucleotide sequences which encode the same amino acids using a different codon from that specifically exemplified in the sequences herein. Such a polynucleotide sequence is referred to herein as an “equivalent” polynucleotide sequence.

The solution further includes variants of the hereinabove described polynucleotides which encode for fragments, such as part or all of the protein, analogs and derivatives of a polypeptide of SEQ ID No. 1 to 13. The variant forms of the polynucleotide may be a naturally occurring allelic variant of the polynucleotide or a non-naturally occurring variant of the polynucleotide. For example, the variant in the nucleic acid may simply be a difference in codon sequence for the amino acid resulting from the degeneracy of the genetic code, or there may be deletion variants, substitution variants and addition or insertion variants. As known in the art, an allelic variant is an alternative form of a polynucleotide sequence, which may have a substitution, deletion or addition of one or more nucleotides that does not substantially alter the biological function of the encoded polypeptide.

The polynucleotides (and corresponding polypeptides) of the proposed solution should be in an isolated form, and preferably they are purified to substantial homogeneity or purity. By substantial homogeneity is meant a purity of at least about 85%. In large scale or industrial applications their use in unpurified form is intended, preferably concentrated by removal of liquid.

The term “isolated” is used to mean that the material has been removed from its original environment (e. g., the natural environment if it is naturally occurring). For example, a naturally occurring polynucleotide or polypeptide present in a living organisms not considered to be isolated, but the same polynucleotide or polypeptide, when separated from substantially all of the coexisting materials in the natural system, is considered isolated. For DNA, the term includes, for example, a recombinant DNA which is incorporated into a vector, into an autonomously replicating plasmid or virus, or into the genomic DNA of a prokaryote or eukaryote; or which exists as a separate molecule (e. g., a cDNA or a genomic or cDNA fragment produced by polymerase chain reaction (PCR) or restriction endonuclease digestion) independent of other sequences. It also includes a recombinant DNA, which is part of a hybrid gene encoding additional polypeptide sequence, e.g., a fusion protein. Further included is recombinant DNA which includes a portion of the nucleotides shown in one of SEQ ID No. 1-13.

In a preferred embodiment, the nucleic acid is a polynucleotide, which has been codon-optimized for recombinant expression in a production host such as yeast.

It is furthermore preferred if a recombinant polynucleotide comprising at least one of the polynucleotide sequence SEQ ID No. 1-13 plus an expression-controlling elements operably linked with said polynucleotide to drive expression thereof is provided.

The object is further solved by providing a polypeptide encoded by at least one of the polynucleotides of SEQ ID No. 1-13, in particular SEQ ID no. 1, 2, 6-12.

Thus, a Methyltransferase and precursor peptide OphMA, a prolyloligopeptidase OphP, P450-type monooxygenases OphM1, OphM2 and acetyltransferase OphC are provided.

Specifically, methyltransferase and precursor peptide OphMA and prolyloligopeptidase OphP are involved in the biosynthesis of omphalotin A while subsequent steps, assigned to P450 monooxygenases and a putative acetyltransferase perform further modifications of the molecule rendering e.g. omphalotin C or D.

The solution also refers to vectors, in particular expression vectors, which include such polynucleotides of SEQ ID No. 1-13.

In particular an expression vector is provided which comprises at least one of the following recombinant polynucleotides:
a polynucleotide encoding for a polypeptide with N-methyltransferase activity and omphalotin precursor peptide activity, consisting essentially of Seq ID No. 1 or 2 or a polynucleotide with at least 70%, preferably at least 90%, more preferably at least 95% identity thereto;
a polynucleotide encoding for a polypeptide with prolyloligopeptidase activity, consisting essentially of Seq ID No. 6 or 7 or a polynucleotide with at least 70%, preferably at least 90%, more preferably at least 95% identity thereto,
a polynucleotide encoding for a polypeptide with monooxygenase activity, consisting essentially of Seq ID No. 8 or a polynucleotide with at least 70%, preferably at least 90%, more preferably at least 95% identity thereto,
polynucleotide encoding for a polypeptide with monooxygenase activity, consisting essentially of Seq ID No. 9 or 10 or a polynucleotide with at least 70%, preferably at least 90%, more preferably at least 95% identity thereto,
a polynucleotide encoding for a polypeptide with acetyltransferase activity, consisting essentially of Seq ID No. 11 or 12 or a polynucleotide with at least 70%, preferably at least 90%, more preferably at least 95% identity thereto, or
a polynucleotide encoding for polypeptides with activity for omphalotin synthesis comprising at least one of the Seq ID No. 1, 2, 6-12, and consisting essentially of Seq ID No. 13 or a polynucleotide with at least 70%, preferably at least 90%, more preferably at least 95% identity thereto.

The vector may be, for example, in the form of a plasmid, a conjugative plasmid, a viral particle, a phage, etc. The vector or the gene may be integrated into the chromosome at a specific or a not specified site. Methods for genome integration of recombinant DNA, such as homologous recombination or transposase-mediated integration, are well known in the art. The engineered host cells can be cultured in conventional nutrient media modified as appropriate for activating promoters, selecting transformants or amplifying the genes of the proposed solution. The culture conditions, such as temperature, pH and the like, are those commonly used with the host cell selected for expression, as well known to the ordinarily skilled artisan.

The polynucleotides of the proposed solution may be employed for producing polypeptides by recombinant techniques. Thus, for example, the polynucleotides may be included in any one of a variety of expression vectors for expressing polypeptides. Such vectors include chromosomal, nonchromosomal and synthetic DNA sequences, e. g. yeast plasmids; bacterial or yeast artificial chromosome (BAC, YAC); yeast episomal or integrative plasmids (YEps, YIps) or a vector suitable for fungi. However, any other vector may be used as long as it is replicable and viable in the host, or can be used for genome integration.

The appropriate DNA sequence may be inserted into the vector by any of a variety of procedures. In general, the DNA sequence is inserted into an appropriate restriction endonuclease site(s) by procedures well known in the art, which procedures are deemed to be within the scope of those skilled in this art.

The DNA sequence in the expression vector is operatively linked to an appropriate expression control sequence(s) (promoter) to direct mRNA synthesis.

The expression vector should also contain a ribosome binding site for translation initiation and a transcription terminator for transcriptional termination. The vector may also include appropriate sequences for amplifying expression. In addition, the expression vectors preferably contain one or more selectable marker genes to provide a phenotypic trait for selection of transformed host cells.

In a most preferred embodiment at least two expression vectors are provided that allow the heterologous expression of at least two of the polynucleotides of Seq ID. No 1-13.

In particular, a first expression vector is provided that enables the expression of a polynucleotide of Seq ID No. 1 or 2 (ophMA) and a second expression vector is provided that enables the expression of a polynucleotide of Seq ID No. 6 or 7 (ophP).

It is however also possible to use one expression vector comprising both ophMA and ophP.

In a preferred embodiment an expression vector for heterologous expression of at least one polynucleotide of SEQ ID. No. 1-13 in yeast, in particular P. pastoris or S. cerevisiae, or in fungi, in particular Aspergillus sp., such as A. niger is provided.

The preferred expression vectors are suitable for heterologous expression of the present polynucleotides/polypeptides in P. pastoris. Such vector may comprise a constitutive, inducible or repressible promoter that can switch on or switch off the polypeptide expression and thus the biosynthesis of omphalotin. Such a suitable promoter may be a methanol inducible promoter such as AOX promoter and is integrated into the genome for chromosomal expression.

An example for P. pastoris expression vector may be expression vector pPIC3.5 K (Thermo Fisher Scientific). Another suitable expression vector for heterologous expression in P. pastoris may be the shuttle vector pPICHOLI-2 (MoBiTech).

In case of S. cerevisiae a Galactose induced system may be used as expression vector or expression system.

As mentioned previously, a host cell that has been genetically engineered by the insertion of a polynucleotide of SEq ID No. 1-13 or the vector containing said polynucleotides is also provided.

The vector containing the appropriate DNA sequence as hereinabove described, as well as an appropriate promoter or control sequence, may be employed to transform the appropriate host to permit the host to express the protein. As representative examples of appropriate hosts, there may be mentioned yeasts such as Pichia and Saccharomyces or fungi such as Aspergillus, P. pastoris and S. cerevisiae and A. niger being the most preferred expression hosts.

In a preferred embodiment, at least one expression vector comprising ophMA and at least one second expression vector comprising ophP are transformed into the host cell. The vectors may be integrated into the chromosome of the host cell or may be present in the host cell as self-replicating vectors.

It is also possible that both genes are expressed together in one (self replicating) vector system or can be integrated together into the chromosome using one vector system.

Thus, in an embodiment a host cell is provided, wherein i) the recombinant polynucleotides are integrated into the chromosome of the host cell, ii) an expression vector comprising at least one or both of the recombinant polynucleotides is integrated into the chromosome of the host cell, or iii) at least one or both of the recombinant polynucleotides is part of a self-replicating vector.

The object is also solved by providing a process for producing at least one omphalotin compound comprising the steps of culturing the host cell containing at least one of the polynucleotides of SEQ ID. No 1-13 under conditions suitable for the biosynthesis of the at least one omphalotin compound.

It is preferred that the biosynthesis of the at least one omphalotin compound is induced by adding at least one suitable inducer to the culture medium.

In a preferred embodiment of the present process the host cell comprises at least one expression vector (or chromosomal expression cassette) with the ophMA encoding sequence and at least one expression vector (or chromosomal expression cassette) with the ophP encoding sequence. The expression of both enzymes OphMA and OphP may be induced by the same or different inducers. The expression of both enzymes enables the biosynthesis of at least one omphalotin compound, in particular omphalotin A.

The present process may further include the step of removing and isolating the at least one omphalotin compound from the culture medium.

The at least one omphalotin compound can be recovered and purified from recombinant cell cultures by methods including salt or solvent precipitation, acid extraction, ultra-filtration, anion or cation exchange chromatography, phosphocellulose chromatography, hydrophobic interaction chromatography, size exclusion chromatography, affinity chromatography, hydroxylapatite chromatography, lectin chromatography and ultrafiltration.

High performance liquid chromatography (HPLC) can be employed for final purification steps. In special cases purification may not be necssary.

### BRIEF DESCRIPTION OF THE DRAWINGS

The proposed solution is further explained in detail by means of the following examples with reference to the figures.

FIG. 1 shows a general overview of the gene cluster involved in the omphalotin biosynthesis.

FIG. 2 shows ion chromatograms of omphalotin A produced in O. olearius.

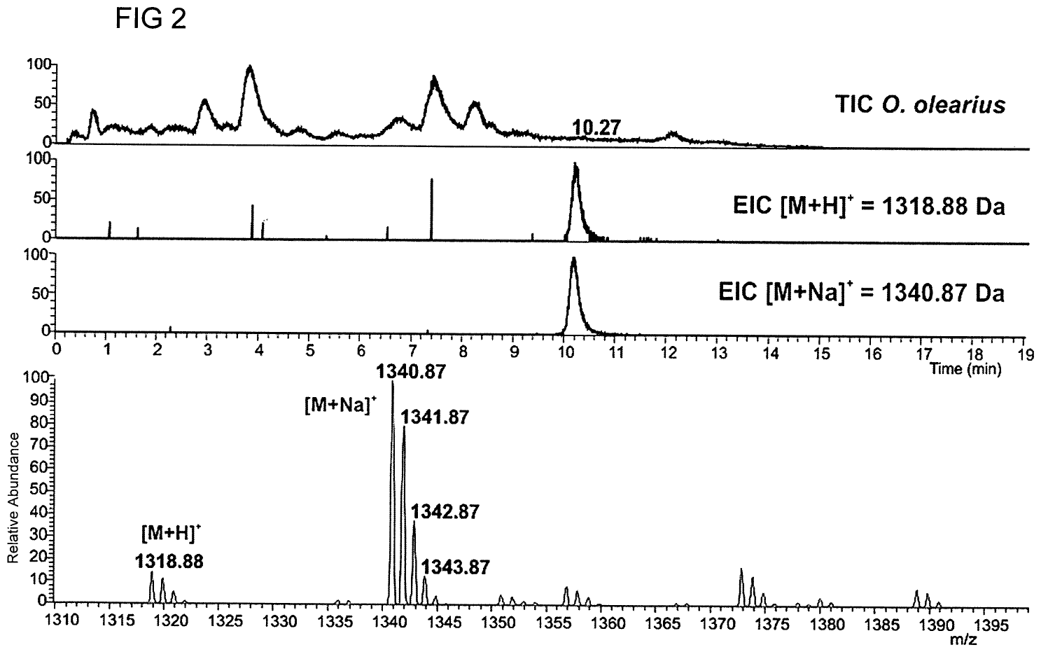

FIG. 3 shows ion chromatograms of omphalotin A intermediate stages produced in P. pastoris.

FIG. 4 shows an MSMS chromatogram of omphalotin A produced by P. pastoris.

FIG. 5 shows an MSMS chromatogram of linear omphalotin A produced by P. pastoris.

FIG. 6 shows Total-ion chromatogram (TIC) and extracted-ion chromatogram (EIC) of cyclized omphalotin A and linear omphalotin A produced by P. pastoris measured by ESI-Orbitrap-MS.

### DETAILED DESCRIPTION

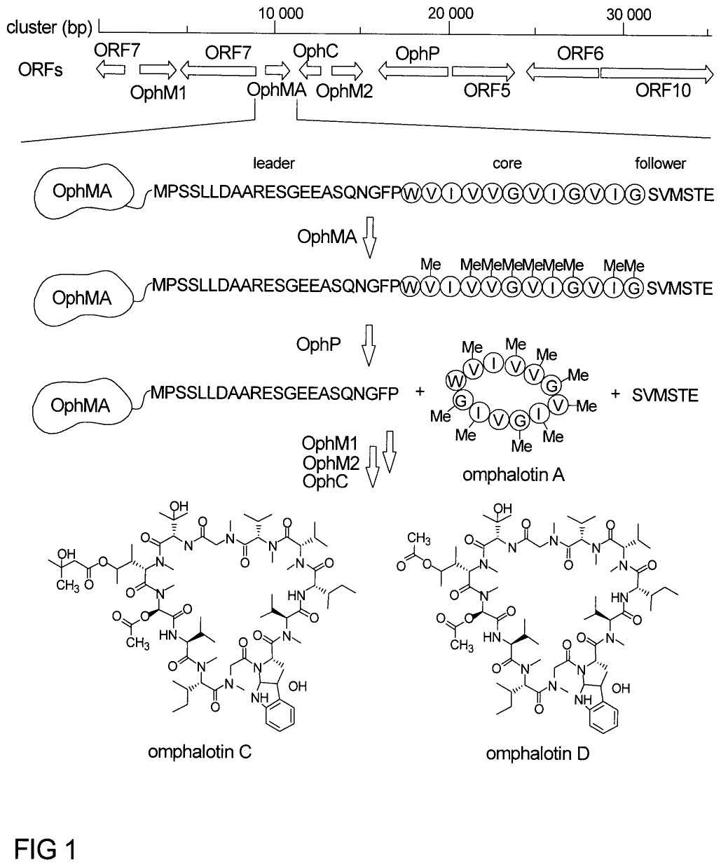

The scheme of FIG. 1 illustrates the gene cluster involved in omphalotin biosynthesis.

The two enzymes, OphMA and OphP, are required to reconstitute the biosynthesis of omphalotin A. Specifically, am automethylation of methyltransferase OphMA is followed by processing of the prolyl oligopeptidase OphP. Subsequent steps introduce hydroxylations (OphM1 and OphM2 as P450 monooxygenases) and acylations by an acetyltransferase (OphC), rendering e.g. omphalotin C and D.

Higher multiply methylated linear omphalotin precursor peptides may also be detected and may be interpreted as the occurrence of shunt products, which may have hydrolyzed from an overwrought or a partially incorrectly folded cyclizing protease OphP. Nevertheless, multiple methylations appear to be a precondition for the action of OphP, which likely forms a peptidylester upon removal of the follower peptide which is then macrocyclized with the N-terminus of the modified core peptide.

The proposed solution represents an initial steps of the omphaplotin biosynthesis, which is characterized by some unusual features: firstly, the striking structural similarity between omphalotin and cyclosporine initially let assume a non-ribosomal peptide assembly. Secondly, multiple N-methylations of the peptide backbone thus far have only been described for NRPS. Thirdly, the key-enzyme OphMA appears to be an automodifying methyltransferase, which requires the subsequent processing by a protease (OphP), a biosynthesis mechanism, which is unprecedented in RiPPs.

### Example 1: Materials and Equipment

All chemicals and antibiotics were purchased from Carl Roth GmbH & Co or Sigma Aldrich and used without further purification unless otherwise specified. Messenger RNA was extracted from Omphalotus olearius strain DSM-3398 (DMSZ—Deutsche Sammlung von Mikroorganismen and Zellkulturen GmbH, Leibniz-Institut, Braunschweig, Germany) using TRIzol® reagent and translated into cDNA with the First Strand cDNA Kit from Thermo Fisher Scientific. Escherichia coli cells DH5α, the overexpression host Pichia pastoris GS115, DNA modifying enzymes (for PCR amplification, restriction digest and ligation) were obtained from Thermo Fisher Scientific as well as the pPIC3.5K vector. The vector pPICHOLI-2 was purchased from MoBiTec. All primers were ordered from Thermo Fisher Scientific and the DNA sequencing was carried out by Eurof ins Genomics (Munich, Germany).

### Example 2: Preparation and Purification of DNA and RNA

mRNA Extraction and cDNA Synthesis

Genes encoding OphMA and OphP were amplified from complementary DNA (cDNA). Therefore messenger RNA (mRNA) from omphalotin producing Omphalotus olearius submerse cultures was extracted. A culture of O. olearius was grown for 7 days (28° C.; 80 rpm) on MEA-medium (30 g malt extract and 3 g soya peptone per liter). Grown colonies were crushed in a mortar and transferred into YMG-medium (10 g malt extract, 4 g yeast extract and 4 g glucose per liter). After an additional incubation of 10 days (28° C.; 80 rpm) O. olearius started to produce Omphalotin. As control for the production of Omphalotin the dodecapeptide was isolated from the supernatant.

Detection of omphalotin was performed by means of HPLC-Orbitrap mass spectrometry (MS) (see S3.2). For the extraction of mRNA with the TRIzol® reagent (Thermo Fisher Scientific) a highly omphalotin producing culture (judged by MS detection) was used and executed according to the manufacturer's instructions. The mRNA was translated into cDNA with the First Strand cDNA Kit (Thermo Fisher Scientific) according the manufacturer's instructions and used as a template for the PCR amplification of ophMA and ophP.

Cloning of ophMA and ophP for Pichia Pastoris

Genes encoding OphMA (methyltransferase+precursor peptide) and OphP (prolyloligopeptidase) of the basidiomycete O. olearius were first amplified by means of PCR from cDNA (0₅ polymerase, NEB) and cloned into the vectors pET28a(+) (ophMA) and pET-trx-1b (ophP). These plasmids were used as template for the amplification and cloning into the vector pPICHOLI-2 (MoBiTech) (ophMA) and vector pPIC3.5K (Thermo Fischer Scientific) (ophP).

Used primers are listed in table S1. For ophP two sets of primers were used in two different

PCR reactions to remove a Sall restriction site (silent mutation). Each PCR reaction (50 μL total volume) consisted of 35 μL sterile ddH₂O, 10 μL 5×Q₅ reaction buffer, 1 μL of 10 μM dNTP solution, 1 μL of each primer (forward and reverse) at 10 μM, 1 μL template and 1 μL of Q₅ polymerase. The PCR reaction was carried out using a T Gradient thermocycler (Biometra). Lid temperature was set to 100° C. and the initial denaturation was at 98° C. for 1 min. The amplification included a 10 s denaturation at 98° C., primer annealing at 68° C. for 10 s and an elongation step at 72° C. for 2 min. The last three steps were repeated 35 times and a final elongation at 72° C. for 5 min followed.

The resulting PCR products were purified with an agarose gel electrophoresis and PCR products were excised and purified using a Gel extraction Kit (Thermo Fisher Scientific) according to manufacturer's instructions. Then the PCR product containing ophMA was digested with SgrDI and NotI, pPICHOLI-2 was digested with SaII and NotI, pPIC3.5K was digested with Sall. Subsequently both plasmids as well as the digested PCR product were purified as described for the PCR products. Both fragments of ophP were cloned into the pPIC3.5K vector using Gibson Assembly® Master Mix (NEB) according the manufacturer's instructions. The gene containing ophMA was cloned with T4 ligase into the pPICHIOLI-2 vector. Thus generated plasmids were used to transform E. coli DH5a cells (Thermo Fischer Scientific) according the manufacturer's instructions. Cells were spread on LB-Agar plates (per liter: 5 g yeast extract, 10 g tryptone, 5 NaCl and 15 g Agar) supplemented with respective antibiotics and incubated over night at 37° C. Single colonies were picked and used to inoculate 20 ml LB medium (per liter: 5 g yeast extract, 10 g tryptone and 5 NaCl) supplemented with respective antibiotics. Cells were cultured overnight at 37° C. and 180 rpm supplemented with respective antibiotics.

Plasmids were isolated using the Plasmid Extraction Kit (Thermo Fischer Scientific) according the manufacturer's instructions. The correct insertion of inserts and DNA sequences was verified by sequencing (Eurofins Genomics). For generation of the heterologous Pichia producer, P. pastoris GS115 cells were first transformed with pPIC3.5K (Thermo Fisher Scientific) bearing ophP according to manufacturer's instructions and in a second step with pPICHOLI-2 (MoBiTech) bearing ophMA according to manufacturer's instructions.

TABLE 1
Used Oligonucleotides and Gene Sequences

| name | Sequence 5′→3′ | Usage |
|---|---|---|
| ophMA fw | CGTCGACGAGAACTTGTATTTCCAGGAGA | ophMA |
| | CTTCCACTCAGACC | |
| ophMA rev | GCGGCCGCATTATTCCGTGCTCATGACTG | ophMA |
| | ATCC | |
| ophP fw | CTAATTATTCGAAGGATCCTACATGAGCG | first |
| | ATAAAATTATTCA | fragment |
| | | ophP |
| Sall rev | AGAACGAAAATATCGTTATCCTGCCGTCG | first |
| | TCCTGATACCA | fragment |
| | | ophP |
| Sall fw | TGGTATCAGGACGACGGCAGGATAACGAT | second |
| | ATTTTCGTTCT | fragment |
| | | ophP |
| ophP rev | GGCCGCCCTAGGGAATTCTACTCAAACTG | second |
| | TTACGCAGGACC | fragment |
| | | ophP |

### Example 3: Purification and Analytical Characterization of Omphalotin

Omphalotin Extraction from Submerse Cultures of O. Olearius and P. Pastoris

### Omphalotus Olearius

For isolation and detection of omphalotin from O. olearius 2 mL supernatant of an omphalotin producing submerse culture (see S2.1) were vigorously mixed for 1 min with the same volume ethyl acetate. The organic phase was separated and vaporized. The resulting pellet was re-suspended in 37% methanol to a final concentration of 0.1 mg /ml and 5 μl were used for HPLC-Orbitrap-MS analysis (see S3.2).

### Pichia Pastoris

For isolation and detection of omphalotin from P. pastoris GS115 a strain carrying ophP integrated into the genome which also carries the pPICHOLI-2 plasmid containing ophMA was used (see S2.2). P. pastoris GS115 cells were pre-cultured overnight at 30° C. in 40 ml YPD-medium (per liter: 20 g peptone, 10 g yeast extract and 20 g glucose) at 160 rpm. The main culture was inoculated with an OD₆₀₀ 0.25 in 500 ml BMMY-medium (per liter: 10 g yeast extract, 10 g trypton, 13.4 g YNB and 100 ml 1 M potassium phosphate buffer adjusted to pH 6.0) and further cultivated at 30° C. and 160 rpm to OD₆₀₀ 1.0 after which gene expression was induced by addition of 0.5% (v/v) methanol. Cells were further incubated at 30° C. and 160 rpm for 72 h and after 24 h and 48 h 0.5% (v/v) methanol was added.

Cells and supernatant (500 ml culture) was separated by centrifugation (30 min, 4° C., 10,000 g). Supernatant was strongly mixed for 1 h with the same volume hexanes. The pellet was re-suspended in 40 ml buffer (300 mM NaCl and 50 mM Tris adjusted to pH 8.0) and disrupted with a French press (TS-series 0.75 kW, Constant systems) at 40 kPsi. Cells debris was centrifuged (60 min, 4° C., 75,000 g) and the supernatant was strongly mixed for 1 h with the same volume of hexanes.

The organic phase was separated and vaporized. The resulting pellets were re-suspended in aqueous methanol (37%) to a final concentration of 0.1 mg /ml and 5 μl were used for HPLC-Orbitrap-MS (see S3.2).

HPLC-ESI-MS Detection of Omphalotin from Submerse Cultures of O. Olearius and P. Pastoris

Detection of omphalotin was conducted using an ESI-Orbitrap-MS (Exactive, Thermo Fisher Scientific) coupled to a HPLC system 1200 (Agilent Technologies). Chromatographic separation were performed using a Poroshell 120 EC.C18 2.7 μm column (50×2.1 mm, Agilent Technologies) with a linear mobile phase gradient consisting of solvent A (0.1% (v/v) formic acid in water and solvent B (0.1% (v/v) formic acid in acetonitrile). The gradient was 37-100% B over 11 min. Data was analyzed using the Thermo Xcalibur 2.2 software.

The production of omphalotin A in O. olearius was analyzed by ion chromatography (see FIG.

2). Total-ion chromatogram (TIC) and extracted-ion chromatogram (EIC) of the cyclized peptide [M+H]⁺=1318.88 Da and [M+Na]⁺=1340.87 Da measured by ESI-Orbitrap-MS.

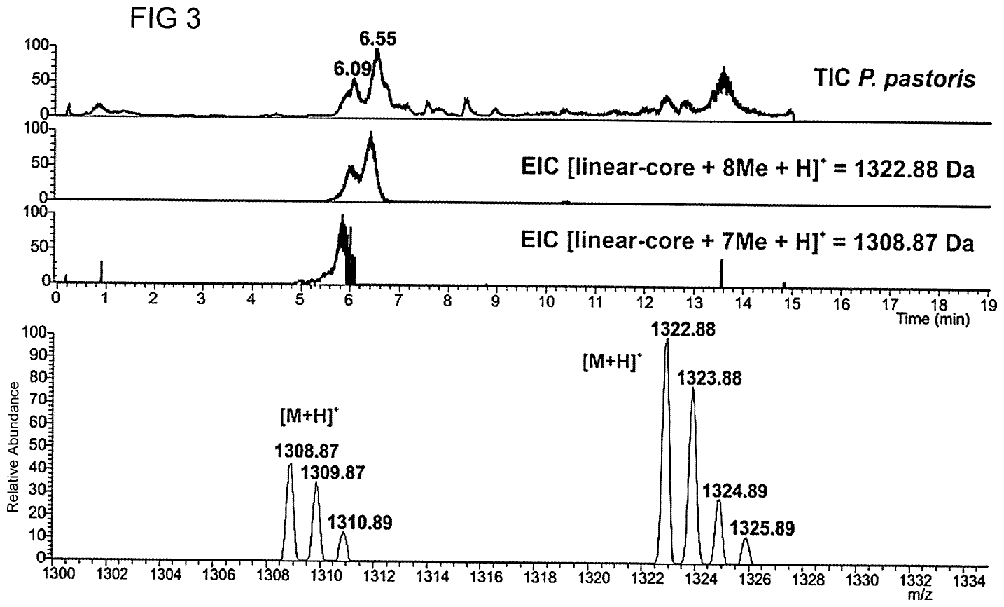

The production of omphalotin A intermediate stages in P. pastoris was also analyzed by ion chromatography (see FIG. 3). Total-ion chromatogram (TIC) and extracted-ion chromatogram (EIC) of the linear core peptide 8× methylated [linear-peptide +8Me+H]⁺=1322.88 Da and the linear core peptide 7× methylated [linear peptide+7 Me+H]⁺=1308.87 Da measured by ESI-Orbitrap-MS.

HPLC-ESI-MS/MS Detection of Omphalotin from Submerse Cultures of O. Olearius and P. Pastoris

All HPLC-ESI-MS/MS analyses were conducted using an ESI-Triple-Quadropol-MS (6460 Series, Agilent Technologies) coupled to a HPLC system 1200 (Agilent Technologies). Chromatographic separations were performed using Grom-Sil 300 Octyl-6MB 2.3 μm columns (50×2.3 mm, Grace) with a linear mobile phase gradient consisting of solvent A (0.1% (v/v) formic acid in water and solvent B (0.1% (v/v) formic acid in acetonitrile). The gradient was 37-100% B over 6 min. Data was analyzed using the MassHunter Qualitative Analysis B.07.00 software.

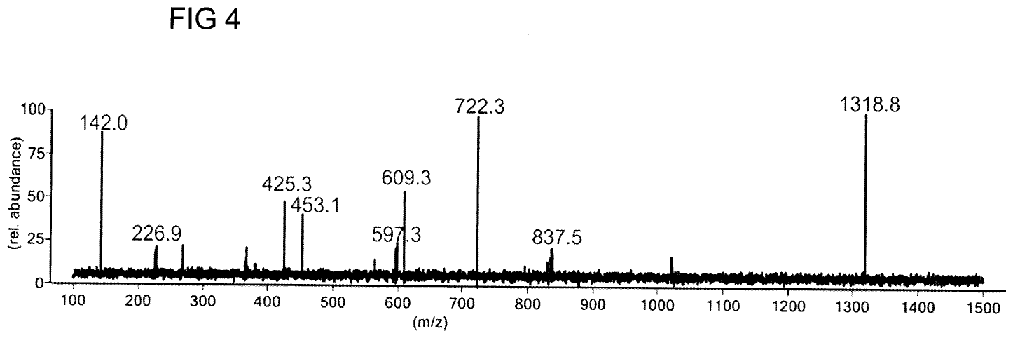

FIG. 4 and Table 2 shows the characteristic and diagnostic fragments of omphalotin A produced by P. pastoris by tandem MS measurements.

TABLE 2
Observed and identified fragments of omphalotin A
after tandem MS measurements

| observed mass | expected mass |
|---|---|
| 142.0 | no match found |
| 226.9 | 227.3 |
| 425.3 | 425.3 |
| 453.1 | 453.3 |
| 597.3 | 597.4 |
| 609.3 | 609.4 |
| 722.3 | 722.5 |
| 837.5 | 837.6 |
| 1318.8 | 1318.9 |

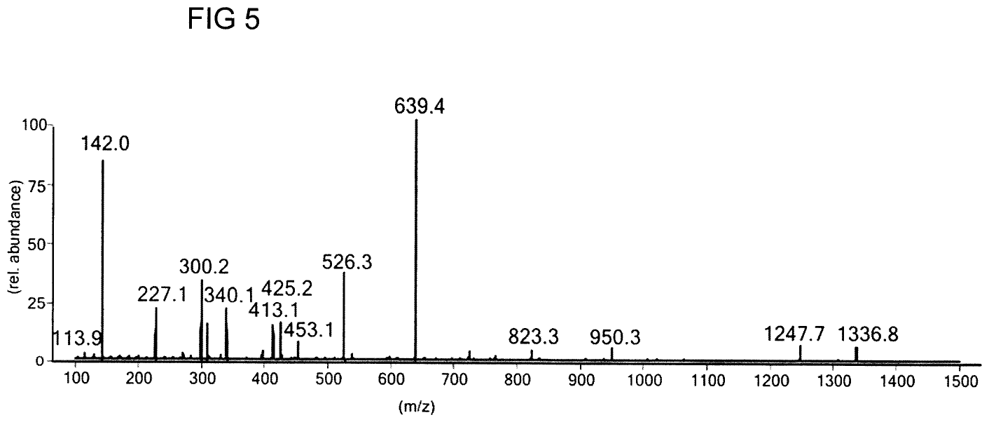

FIG. 5 and Table 3 show the structural characterization of linear omphalotin A produced by P. pastoris by tandem MS measurements.

TABLE S3
Observed and identified fragments of
linearomphalotin A after tandem MS measurements

| observed mass | expected mass |
|---|---|
| 113.9 | 114.1 |
| 142.0 | no match found |
| 227.1 | 227.2 |
| 300.2 | 300.2 |
| 340.1 | 340.3 |
| 413.1 | 413.3 |
| 425.2 | 425.3 |
| 453.1 | 453.3 |
| 526.3 | 526.4 |
| 639.4 | 639.4 |
| 823.3 | 823.5 |
| 950.3 | 950.6 |
| 1247.7 | 124.9 |
| 1336.8 | 1336.9 |

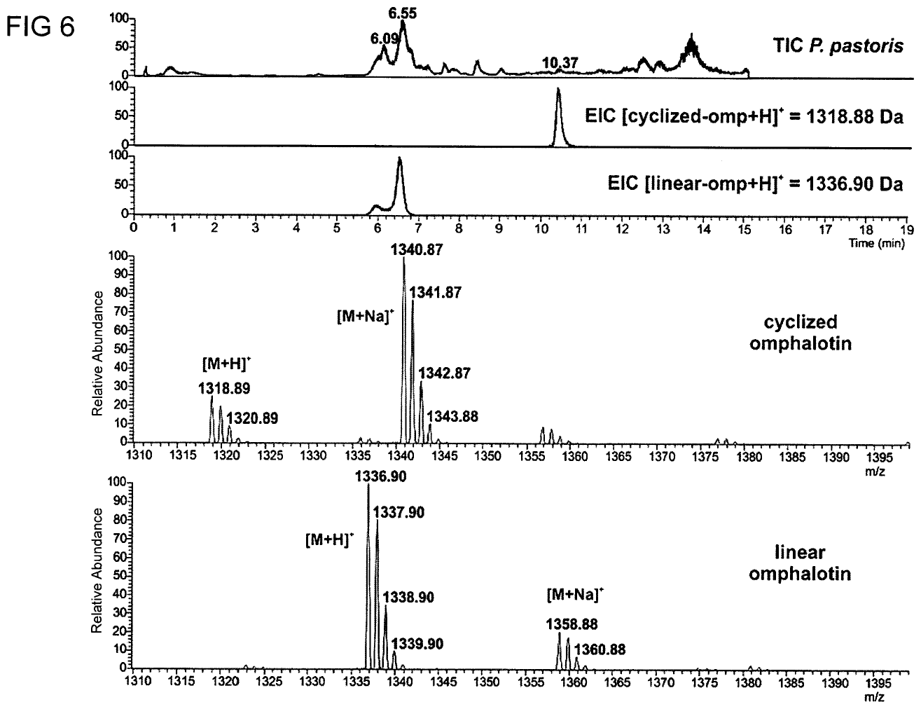

In FIG. 6 the total-ion chromatogram (TIC) and extracted-ion chromatogram (EIC) of cyclized omphalotin A and linear omphalotin A produced by P. pastoris measured by ESI-Orbitrap-MS is another proof of the successful synthesis.

1. A host cell for the heterologous synthesis of at least one omphalotin compound comprising the following recombinant polynucleotides:
a first polynucleotide encoding for a polypeptide with N-methyltransferase activity and omphalotin precursor peptide activity, consisting essentially of Seq ID No. 1 or 2 or a polynucleotide with at least 70%, preferably at least 90%, more preferably at least 95% identity thereto;
a second polynucleotide encoding for a polypeptide with prolyloligopeptidase activity, consisting essentially of Seq ID No. 6 or 7 or a polynucleotide with at least 70%, preferably at least 90%, more preferably at least 95% identity thereto, and
expression-controlling elements operably linked with said polynucleotides to drive expression thereof in the host cell.

2. The host cell according to claim 1, wherein the host cell is selected from yeast, in particular from Pichia or Saccharomyces, or from a fungi, in particular Aspergillus.

3. The host cell according to claim 1, wherein the host cell is selected from Pichia pastoris.

4. The host cell according to claim 1, further comprising a polynucleotide encoding for a polypeptide with monooxygenase activity, consisting essentially of Seq ID No. 8 or a polynucleotide with at least 70%, preferably at least 90%, more preferably at least 95% identity thereto.

5. The host cell according to claim 1, further comprising a polynucleotide encoding for a polypeptide, in particular with monooxygenase activity, consisting essentially of Seq ID No. 9 or 10 or a polynucleotide with at least 70%, preferably at least 90%, more preferably at least 95% identity thereto.

6. The host cell according to claim 1, further comprising a polynucleotide encoding for a polypeptide with acetyltransferase activity, consisting essentially of Seq ID No. 11 or 12 or a polynucleotide with at least 70%, preferably at least 90%, more preferably at least 95% identity thereto.

7. The host cell according to claim 1, further comprising a polynucleotide encoding for polypeptides with activity for omphalotin synthesis comprising at least one of the Seq ID No. 1-12, and consisting essentially of Seq ID No. 13 or a polynucleotide with at least 70%, preferably at least 90%, more preferably at least 95% identity thereto.

8. The host cell according to claim 1, wherein the recombinant polynucleotides are integrated into the chromosome of the host cell.

9. The host cell according to claim 1, wherein an expression vector comprising at least one of the recombinant polynucleotides is integrated into the chromosome of the host cell.

10. The host cell according to claim 1, wherein at least one of the recombinant polynucleotides is part of a self-replicating vector.

11. An expression vector comprising at least one of the following recombinant polynucleotides:
a polynucleotide encoding for a polypeptide with N-methyltransferase activity and omphalotin precursor peptide activity, consisting essentially of Seq ID No. 1 or 2 or a polynucleotide with at least 70%, preferably at least 90%, more preferably at least 95% identity thereto;
a polynucleotide encoding for a polypeptide with prolyloligopeptidase activity, consisting essentially of Seq ID No. 6 or 7 or a polynucleotide with at least 70%, preferably at least 90%, more preferably at least 95% identity thereto,
a polynucleotide encoding for a polypeptide with monooxygenase activity, consisting essentially of Seq ID No. 8 or a polynucleotide with at least 70%, preferably at least 90%, more preferably at least 95% identity thereto,
polynucleotide encoding for a polypeptide with monooxygenase activity, consisting essentially of Seq ID No. 9 or 10 or a polynucleotide with at least 70%, preferably at least 90%, more preferably at least 95% identity thereto,
a polynucleotide encoding for a polypeptide with acetyltransferase activity, consisting essentially of Seq ID No. 11 or 12 or a polynucleotide with at least 70%, preferably at least 90%, more preferably at least 95% identity thereto, or
a polynucleotide encoding for polypeptides with activity for omphalotin synthesis comprising at least one of the Seq ID No. 1, 2, 6-12, and consisting essentially of Seq ID No. 13 or a polynucleotide with at least 70%, preferably at least 90%, more preferably at least 95% identity thereto.

12. The expression vector according to claim 11 for heterologous expression of at least one of the polynucleotides in yeast, in particular in Pichia or Saccharomyces, or in fungi, in particular in Aspergillus.

13. The expression vector according to claim 11 for heterologous expression of at least one of the polynucleotides in the yeast Pichia pastoris.

14. The expression vector according to claim 11, wherein the vector is integrated into the chromosome of the yeast or fungi.

15. The expression vector according to claim 11, wherein the vector is a self-replicating vector.

16. A process for producing at least one omphalotin compound comprising the step of culturing the host cell of claim 1 under conditions suitable for the biosynthesis of the at least one omphalotin compound.

17. The process according to claim 16, wherein the biosynthesis of the at least one omphalotin compound is induced by adding at least one suitable inducer to the culture medium.

18. The process according to claim 16, further including the step of removing and isolating the at least one omphalotin compound from the culture medium.
